(19) United States
(12) Patent Application Publication
(10) Pub. No.: US 2025/0268934 A1
(43) Pub. Date: Aug. 28, 2025

(54) Compositions and Methods for the Treatment of Bacterial Vaginosis

(71) Applicants: Wayne State University, Detroit, MI (US); Aolis Investment AG, Zug (CH)

(72) Inventors: Jack D. Sobel, West Bloomfield, MI (US); Koral Embil, Yalikavak / Bodrum (TR)

(73) Assignees: Wayne State University, Detroit, MI (US); Aolis Investment AG, Zug (CH)

(21) Appl. No.: 18/856,866

(22) PCT Filed: Apr. 14, 2023
(86) PCT No.: PCT/US2023/065786

§ 371 Date: Oct. 14, 2024

Related U.S. Application Data
(60) Provisional application No. 63/363,038, filed on Apr. 15, 2022.

(51) Int. Cl. A61K 33/22; A61K 9/00; A61K 31/4164; A61P 31/04

(52) U.S. Cl. CPC A61K 33/22; A61K 9/0036; A61K 31/4164; A61P 31/04

(57) ABSTRACT

Provided herein are methods of treating vaginitis by simultaneous local application per vagina of both boric acid and nitroimidazole. Also provided are compositions useful in such treatments, for instance containing 100-1,000 mg of nitroimidazole and 300-900 mg boric acid. Vaginal application of nitroimidazole allows a higher but safe dose to be administered simultaneously with boric acid for the effective treatment of bacterial vaginosis (BV). Also provided are methods for prophylactic treatment of patients, such as those prone to recurrent BV.

### CROSS-REFERENCE TO RELATED APPLICATIONS

This U.S. National Stage application claims priority to and the benefit of the earlier filing of International Patent Application No. PCT/US2023/065786, filed Apr. 14, 2023. International Patent Application No. PCT/US2023/065786 claims priority to U.S. Provisional Application No. 63/363,038, filed on Apr. 15, 2022. Each of these earlier-filed applications is incorporated by reference herein in its entirety.

### FIELD OF THE DISCLOSURE

The present disclosure relates to the treatment of bacterial vaginosis and compositions for use in such treatment. In particular, the disclosure relates to the treatment of refractory or recurrent bacterial vaginosis.

### BACKGROUND OF THE DISCLOSURE

Vaginitis is an infection of the vagina and vulva and is a common gynecological condition encountered by physicians. Vulvovaginal symptoms include itching, burning, irritation, and abnormal discharge. The vast majority of cases of vaginitis are caused by infections, specifically bacterial vaginosis (BV) (40-50%), vulvovaginal candidiasis (VVC) (20-25%), and trichomonal vaginitis (15-20%). Diagnosis of vaginitis includes physical examination, measuring the pH of the vaginal discharge, microscopy (mostly by vaginal wet mount), and culture of the discharge.

Bacterial vaginosis represents a profound shift in the vaginal microbiota and is routinely diagnosed according to Amsel's clinical criteria. BV is characterized by high bacterial species diversity; increased loads of facultative anaerobes, including Gardnerella vaginalis, Prevotella spp., Atopobium vaginae, and other fastidious BV-associated bacteria, such as Megasphaera, Sneathia, and Clostridiales species; increased production of volatile amines; and a rise in vaginal pH to >4.5. This change is accompanied by a marked depletion of key Lactobacillus species such as Lactobacillus crispatus, which produces lactic acid, bacteriocins, and other antimicrobial molecules, and which is thought to play an important role in host defense against pathogens.

BV is the most common cause of vaginitis worldwide and mostly affects women of reproductive age. Global prevalence of BV in the general population ranges between 23-29% (Peebles et al., Sex Transm Dis, 46(5): 304-311, 2019). It is responsible for considerable and persistent discomfort and, if left untreated, can lead to serious complications such as chorioamnionitis, pre-term labor and enhanced susceptibility to sexually transmitted diseases including HIV infection, N. gonorrhoeae, C. trachomatis and HSV-2.

BV is extremely difficult to treat and cure. FDA-approved treatment options include oral or topical metronidazole, oral tinidazole, and oral or topical clindamycin. However, such treatment frequently fails leading to a high rate of refractory disease as well as a high recurrence rate. This represents a major challenge to effective therapy and many women may be prescribed 5-6 courses of repeated therapy annually. Treatment options for uncomplicated as well as recurrent BV (RBV) are limited and remain unsatisfactory. Women suffering from intractable and frequent recurrences, in particular, are ill-served by available treatment options. Although antimicrobial therapy is effective short-term in relieving symptoms, BV is associated with high post-treatment recurrence rates (30% in 3 months, 30-50% within 6 months, and rising to 80% within one year). In the absence of curative therapy, physicians resort to treating each individual episode. About 15-20% of patients also remain refractory to current treatment options, i.e. they do not respond to treatment.

Despite its global prevalence and significance by virtue of multiple complications, a fundamental understanding of BV disease pathophysiology is still not available. Considerable progress in understanding BV transmission and microbiology has been made, resulting in new diagnostic methodologies. However, therapeutic advances have not been forthcoming. Standard-of-care drug treatment recommendations and guidelines have not substantially changed in the last three decades. Adding to the frustration of treating refractory intractable symptomatic disease, women frequently present in the clinic with recurrent symptomatic episodes, often more than 4 attacks annually, and few therapeutic options are available. The emotional and economic consequences of recurrent BV are enormous and a source of considerable frustration to both physicians and women. Clinicians worldwide are faced by women plagued by recurring symptoms with manifestation playing havoc with their self-esteem and sexual lives. The medical community has no current answer other than repeat prescriptions.

Previous studies have evaluated a long-term maintenance antimicrobial regimen of twice weekly vaginal metronidazole 0.75% gel (Sobel et al., Am J Obstet Gynecol, 194: 1283-1289, 2006). Continued use of this maintenance regimen was reasonably effective in controlling RBV, i.e., preventing symptomatic BV recurrence, but only while prophylaxis continued, and long-term efficacy or cure was uncommon. This suppressive regimen was still accompanied by a disappointing recurrence rate when the maintenance regime was discontinued. Although the use of maintenance vaginal metronidazole twice weekly is now widely used to control or prevent RBV, it does not provide a cure.

Reichman et al. (Sex Transm Dis, 36: 732-734, 2009) treated patients with RBV twice daily for 7 days with standard oral dose nitroimidazole therapy (500 mg metronidazole or tinidazole), followed by 21 days of intravaginal boric acid (600 mg/day). Patients determined to be in remission were subsequently treated with maintenance suppressive metronidazole gel twice weekly for 16 weeks. Topical vaginal boric acid given daily after conventional oral metronidazole and followed by maintenance suppressive metronidazole gel resulted in breakthrough infections during the metronidazole gel maintenance therapy of 12%, with recurrence of approximately 66% by 28 weeks. The Sobel et al. (Sobel et al., Am J Obstet Gynecol, 194: 1283-1289, 2006) study (using twice weekly metronidazole vaginal gel) is described as resulting in 25% breakthrough infections with recurrence of 66% at 32 weeks. Reichman et al. hypothesized that boric acid may remove the BV-related biofilm.

In EP 2 529 723, a retrospective case review of the sequential treatment protocol conducted by Reichman et al. (Sex Transm Dis, 36: 732-734, 2009) (i.e., nitroimidazole orally, followed by boric acid intravaginally) is reported. After a total treatment time of 6 months, the report notes that only 77% of patients were clinically cured, i.e., asymptomatic and negative for Amsel's clinical criteria for BV. Only 67% of patients remained cured at 9 months, i.e., 3 months after maintenance therapy. The median duration of remission was 8.7 months. Based on the hypothesis that boric acid has therapeutically-relevant biofilm disrupting properties, EP 2 529 723 suggests the combined use of boric acid with ethylene diamine tetra-acetic acid (EDTA). It demonstrates a synergistic effect for this combination of agents against C. albicans and G. vaginalis biofilms in vitro and proposes that boric acid should be used together with EDTA to enhance its biofilm disrupting properties. Compositions for topical application to the vagina and/or the vulva that contain therapeutically effective amounts of boric acid and EDTA are therefore proposed to treat vaginal infection and pathogenic vaginal biofilms.

More recently, simultaneous administration of oral nitroimidazole and boric acid therapy followed by long-term twice weekly vaginal metronidazole gel for 5 months was used to treat recurrent BV (Surapaneni et al., Sex Transm Dis, 48(10): 761-765, 2021). The treatment regimen consisted of oral tinidazole or metronidazole 500 mg twice daily for 7 days starting on the same day as vaginal boric acid (600 mg) which was given for 30 days. Follow-up visits occurred on day 30 to 35 and, if asymptomatic and with <3 Amsel criteria for BV, twice weekly metronidazole gel 0.75% was then prescribed for 5 months. The initial regimen of nitroimidazole and simultaneous but prolonged vaginal boric acid was reported to achieve a satisfactory response in 87 out of 88 patients. Thereafter, the maintenance metronidazole gel prevented symptomatic BV recurrence in 70% of compliant patients. Long-term cure at a 12-month follow up was demonstrated in about 50% of the patient population. Duration of the initial treatment (30 days) and the need for long-term maintenance therapy resulted in frequent patient follow-up loss. In compliant patients, recurrence rates of BV were still significant.

Despite these developments, there remains a need for an alternative treatment for bacterial vaginosis. In particular, there remains an urgent need for an effective treatment which can at least control and potentially cure recurrent BV.

### SUMMARY OF THE DISCLOSURE

Previous studies discussing the treatment of vaginitis, such as the study reported by Surapaneni et al. (Sex Transm Dis, 48(10): 761-765, 2021), have been sub-optimal in giving oral tinidazole or metronidazole therapy to patients in combination with vaginal boric acid. The two active components, although simultaneous, were administered by different routes. As the inventors have recognized, administration of an optimal dose of tinidazole or metronidazole in the Surapaneni study was not possible due to their poor tolerability (i.e., toxicity) when delivered orally.

Provided herein are methods of treating vaginitis by simultaneous local application per vagina of both boric acid and nitroimidazole. In embodiments, this method is highly beneficial in eradicating the bacterial biofilm and pathogens, and thus provides not only short-term (e.g., 30 day) but also long-term control of recurrent bacterial vaginosis (BV), for example beyond the 30-day limitation in previously approved products. Specifically, vaginal application of the nitroimidazole allows a higher but safe dose to be administered simultaneously with boric acid for the effective treatment of BV. In contrast to previous studies with boric acid involving prolonged therapy for 21-30 days, it is expected that the optimal duration of the combination therapy will be much shorter, for example only 7 days, leading to much improved patient compliance in completing the course of the treatment. In embodiments, methods provided herein include the prophylactic treatment of patients prone to recurrent BV.

Provided herein is a vaginal composition that combines nitroimidazole and boric acid in a stable formulation and, advantageously, allows administration of a higher dose of a nitroimidazole (e.g., metronidazole) in a single dose unit. This provides a convenient, well tolerated, high dose of the nitroimidazole for simultaneous administration in combination with boric acid. In embodiments, the vaginal composition is provided in the form of a vaginal suppository for ease of self-administration by the patient.

In vitro studies documented herein confirm the synergistic effect of combining metronidazole or tinidazole (exemplar nitroimidazole drugs) with boric acid in inhibiting the growth of planktonic cells and inhibiting biofilms of Gardnerella vaginalis, the dominant bacterial pathogen responsible for BV. These results support the notion that the combination of a nitroimidazole and boric acid can provide an improved treatment for bacterial vaginosis, in particular a treatment that is short in duration (thereby maximizing patient compliance), yet highly effective in eradicating the disease and in reducing post-treatment recurrence rates. In embodiments, the compositions and methods herein are used in refractory patients that fail to respond to current treatment options.

In one aspect, provided herein is a method for the treatment of bacterial vaginosis in which boric acid and a nitroimidazole active against Gardnerella vaginalis (G. vaginalis) are simultaneously administered to the patient by vaginal application. In one embodiment, the nitroimidazole is a 5-nitroimidazole or a pharmaceutically acceptable salt thereof. In one embodiment, the 5-nitroimidazole is selected from metronidazole and tinidazole. In some embodiments, the nitroimidazole is present in the composition in an amount from 4 to 40 wt. % based on the total weight of the composition. In some embodiments, the boric acid is present in the composition in an amount of from 10 to 30 wt. % based on the total weight of the composition. In an embodiment, the composition additionally includes an active agent effective against Candida albicans. In an embodiment, the active agent effective against Candida albicans is an imidazole or a pharmaceutically acceptable salt thereof. In some embodiments, the imidazole is selected from miconazole, tioconazole, and pharmaceutically acceptable salts thereof. In some embodiments, the active agent effective against Candida albicans is present in the composition in an amount of from 2 to 10 wt. % based on the total weight of the composition. In an embodiment, the composition is substantially free from ethylene diamine tetra acetic acid (EDTA). In an embodiment, the composition is provided in the form of a cream or a vaginal suppository. In an embodiment, the vaginal suppository is a vaginal ovule including a hard fat pessary base. In some embodiments, the vaginal suppository contains from 100 to 1,000 mg of the nitroimidazole and from 300 to 900 mg boric acid. In some embodiments, the vaginal suppository contains from 500 to 1,000 mg metronidazole. In some embodiments, the vaginal suppository contains from 100 to 400 mg tinidazole. In an embodiment, the patient is a female having recurrent bacterial vaginosis. In some embodiments, the method is effective to prevent recurrence of bacterial vaginosis in the patient for a period of at least 30 days, preferably at least 45 days, e.g., at least 60 days from the start of the treatment. In an embodiment, the treatment is effective to prevent recurrence of bacterial vaginosis in the patient without the need for conventional antibiotic maintenance therapy. In an embodiment, the patient is a female patient having refractory bacterial vaginosis. In an embodiment, the patient is refractory to an FDA-approved treatment for bacterial vaginosis, for example oral or vaginal treatment with clindamycin or metronidazole. In some embodiments, the method includes administration of the composition for a treatment period of up to 14 days, preferably up to 10 days, e.g., up to 7 days. In one embodiment, the treatment period is 7 days. In one embodiment, the composition is administered once a day. In one embodiment, the composition is provided in the form of a vaginal suppository that contains 750 mg metronidazole and 600 mg boric acid, and the method includes administration of the vaginal suppository once a day for a period of 7 days.

In one aspect, the disclosure provides a method of treating bacterial vaginosis in a patient in need thereof, the method including vaginal administration to the patient of a composition including a nitroimidazole active against G. vaginalis and boric acid, wherein a therapeutically effective amount of the composition is administered.

In one aspect, the disclosure provides a composition including a nitroimidazole active against G. vaginalis and boric acid for use in a method of treating bacterial vaginosis in a patient, the method including intravaginal administration of the composition to the patient, wherein a therapeutically effective amount of the composition is administered.

In one aspect, the disclosure provides the use of a composition including a nitroimidazole active against G. vaginalis and boric acid in the manufacture of a medicament for use in a method of treating bacterial vaginosis in a patient, wherein the medicament is intravaginally administered to the patient and wherein a therapeutically effective amount of the composition is administered.

In one aspect, the disclosure provides a pharmaceutical composition for vaginal administration including a nitroimidazole active against G. vaginalis and boric acid, together with at least one pharmaceutically acceptable excipient suitable for vaginal administration. In one embodiment, the nitroimidazole is a 5-nitroimidazole or a pharmaceutically acceptable salt thereof. In one embodiment, the 5-nitroimidazole is selected from metronidazole and tinidazole. In some embodiments, the nitroimidazole is present in the pharmaceutical composition in an amount from 4 to 40 wt. % based on the total weight of the composition. In some embodiments, the boric acid is present in the pharmaceutical composition in an amount of from 10 to 30 wt. % based on the total weight of the composition. In one embodiment, the pharmaceutical composition additionally includes an active agent effective against Candida albicans. In one embodiment, the active agent effective against Candida albicans is an imidazole or a pharmaceutically acceptable salt thereof. In some embodiments, the imidazole is selected from miconazole, tioconazole, and pharmaceutically acceptable salts thereof. In some embodiments, the active agent effective against Candida albicans is present in the pharmaceutical composition in an amount of from 2 to 10 wt. % based on the total weight of the composition. In one embodiment, the pharmaceutical composition is substantially free from ethylene diamine tetra acetic acid (EDTA). In one embodiment, the pharmaceutical composition is provided in the form of a cream or a vaginal suppository. In one embodiment, the vaginal suppository is a vaginal ovule including a hard fat pessary base. In some embodiments, the vaginal suppository contains from 100 to 1,000 mg of the nitroimidazole and from 300 to 900 mg boric acid. In some embodiments, the vaginal suppository contains from 500 to 1,000 mg metronidazole. In some embodiments, the vaginal suppository contains from 100 to 400 mg tinidazole. In some embodiments, the pharmaceutical is provided in the form of a vaginal suppository that contains 750 mg metronidazole and 600 mg boric acid.

In one aspect, the disclosure provides a kit including: (i) a pharmaceutical composition including a nitroimidazole active against G. vaginalis and boric acid; (ii) an applicator adapted for delivery of the composition to the vaginal cavity; and optionally (iii) instructions for the vaginal administration of the pharmaceutical composition in the treatment of bacterial vaginosis.

Also provided is a method of treating bacterial vaginosis in a female patient in need thereof, including administering vaginally to the patient: 100 to 1,000 mg of nitroimidazole compound(s) active against Gardnerella vaginalis; and 300 to 900 mg boric acid. In examples, the nitroimidazole compound(s) include at least one of: 500 to 1,000 mg metronidazole; or 100 to 400 mg tinidazole.

Another embodiment is a therapeutic composition, including: 100 to 1,000 mg of nitroimidazole compound(s) active against Gardnerella vaginalis and 300 to 900 mg boric acid, wherein the composition is formulated for vaginal administration.

Yet another embodiment is a method of treating bacterial vaginosis in a patient in need thereof, the method including administering vaginally to the patient a composition including: at least one nitroimidazole compound active against Gardnerella vaginalis; and boric acid, wherein a therapeutically effective amount of the composition is administered.

Also described are compositions including at least one nitroimidazole compound active against G. vaginalis and boric acid for use in any of the methods of treating bacterial vaginosis described herein.

Use of a composition including at least one nitroimidazole compound active against G. vaginalis and boric acid in the manufacture of a medicament for use in a method of treating bacterial vaginosis as described herein is also provided.

Another embodiment is a kit including: a composition including at least one nitroimidazole compound active against G. vaginalis and boric acid, and an applicator adapted for delivery of the composition to a vaginal cavity of a subject.

Also provided are pharmaceutical compositions formulated for vaginal administration, including: at least one nitroimidazole compound active against Gardnerella vaginalis, boric acid, and at least one pharmaceutically acceptable excipient.

### BRIEF DESCRIPTION OF THE DRAWINGS

FIG. 1. Isobologram for the combination treatment of boric acid and metronidazole against planktonic cells of G. vaginalis 14018.

FIG. 2: Isobologram for the combination treatment of boric acid and tinidazole against planktonic cells of G. vaginalis 14018.

FIG. 3: Relative biofilm mass following combination treatments of boric acid and metronidazole for the inhibition of biofilm formation by G. vaginalis ATCC 14018. Single drug treatments are shown (BA MIC-B₉₀ and metronidazole MIC-B₉₀) and compared to effective combination treatments (as determined by FICI₉₀ calculations). Concentrations for the displayed FIC₉₀ values are marked with * in Table 6.

FIG. 4: Relative biofilm mass following combination treatments of boric acid and tinidazole for the inhibition of biofilm formation by G. vaginalis ATCC 14018. Single drug treatments are shown (BA MIC-B₉₀ and tinidazole MIC-B₉₀) and compared to effective combination treatments (as determined by FICI₉₀ calculations). Concentrations for the displayed FIC₉₀ values are marked with * in Table 7.

FIG. 5: Isobologram for the combination of boric acid and metronidazole against biofilm formation by G. vaginalis 14018.

FIG. 6: Isobologram for the combination of boric acid and tinidazole against biofilm formation by G. vaginalis 14018.

FIG. 7: Isobologram for the combination of boric acid and metronidazole against biofilm-associated G. vaginalis 14018.

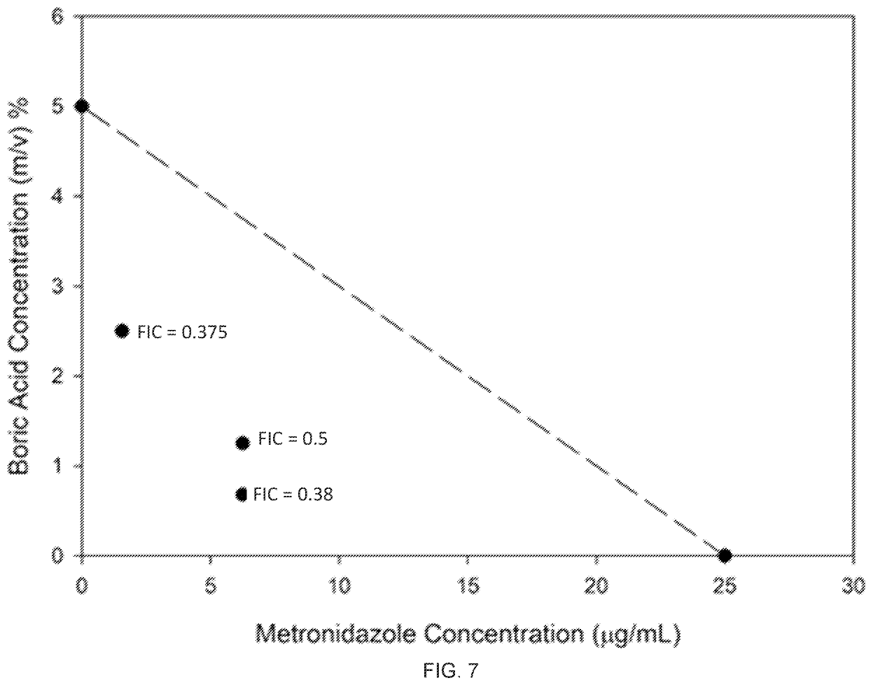

FIG. 8: Isobologram for the combination of boric acid and tinidazole against biofilm-associated G. vaginalis 14018.

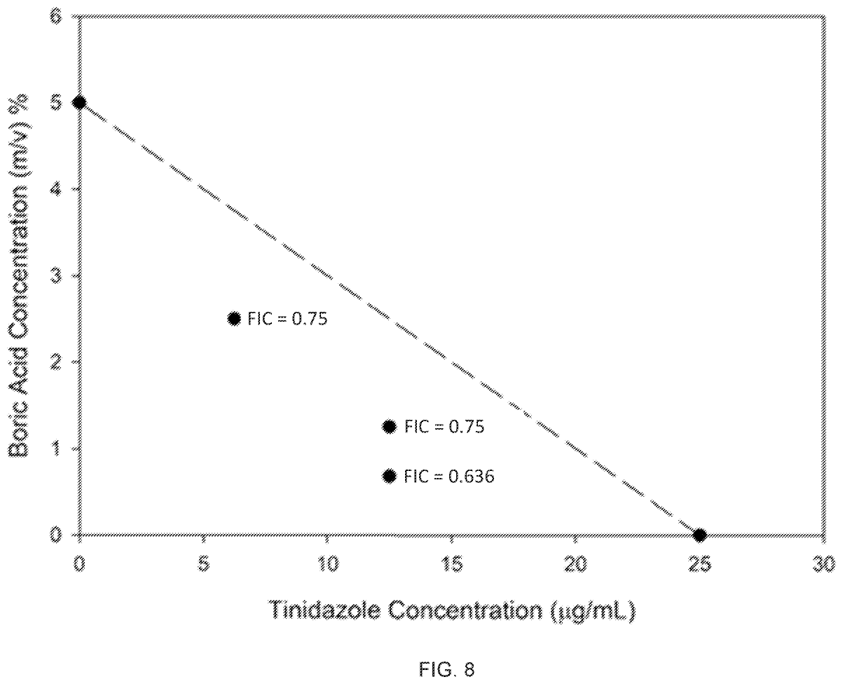

### DETAILED DESCRIPTION

The treatment method herein described involves vaginal administration of a composition containing boric acid and a nitroimidazole (or a mixture of two or more nitroimidazole compounds) effective against Gardnerella vaginalis. Though not wishing to be bound by theory, it is postulated that the boric acid is effective to damage or destroy the bacterial biofilm and to act as a bacteriostatic agent in reducing the presence and/or number of bacteria responsible for the disease. The nitroimidazole(s) simultaneously eradicates the microbial cause of bacterial vaginosis (Gardnerella vaginalis and other anaerobic pathogens). Where the nitroimidazole(s) (e.g., metronidazole) may cause any secondary candidal infection, such as VVC, the boric acid may additionally prevent and/or treat this thereby avoiding the need to use a second therapeutic agent (e.g., fluconazole) to treat this. The active components of the composition have different mechanisms of action against bacterial vaginosis which leads to a higher potency in treatment. Significantly, administration of a composition containing both active agents (or the two active agents otherwise administered concomitantly or simultaneously, even if in different compositions) provides an enhanced synergistic effect in treatment of the condition and is effective to reduce (e.g., to prevent) its recurrence.

Nitroimidazoles effective against G. vaginalis are well known in the art, for example in the treatment of vaginitis. Any such compounds, including derivatives thereof and mixtures of two or more thereof, may be used in the compositions and methods disclosed herein. Derivatives include the pharmaceutically acceptable salts. Nitroimidazole antibiotics are classified according to the position of the nitro group on the imidazole ring. 5-nitroimidazoles are particularly suitable for use in the compositions and methods disclosed herein.

Nitroimidazole compounds that may be used in the compositions and methods disclosed herein include, but are not limited to, metronidazole, tinidazole, nimorazole, dimetridazole, pretomanid, ornidazole, megazol, azanidazole, secnidazole, benznidazole, and mixtures of two or more thereof (e.g., mixed in any proportion). Examples of 5-nitroimidazoles that find use in the compositions and methods disclosed herein include metronidazole and tinidazole, or a mixture of the two compounds. In exemplary embodiments, the nitroimidazole effective against G. vaginalis is metronidazole. Metronidazole is also effective against Trichomonas vaginalis.

The composition for use in a method disclosed herein may be provided in any form suitable for intravaginal administration. For example, it may be provided in the form of an ointment, cream, a vaginal suppository, a solution, a suspension, a gel, or a foam. The composition may also be contained within a vaginal ring, tampon, or sponge, for example. In embodiments, the composition is provided in the form of a cream or a vaginal suppository. Such administration forms are particularly convenient for self-administration by the patient to the vagina and/or the vulva.

As used herein, the term “vaginal suppository” refers to any solid unit dosage form adapted for insertion into the vagina and includes a vaginal ovule, vaginal tablet and vaginal capsule. A vaginal suppository may be delivered to the vaginal cavity using a special applicator or it may be self-administered via fingertip insertion.

The term “vaginal ovule” refers to a solid unit dosage form adapted for insertion into the vagina which contains the active agents in a pessary base. The term “vaginal tablet” refers to a solid unit dosage form adapted for insertion into the vagina which contains a mixture of the active agents and excipients pressed or compacted from a powder. Suitable excipients to produce vaginal tablets are well known in the art and include diluents, binders, granulating agents, glidants, and lubricants to aid in tabletting. A vaginal tablet may include a coating, for example a polymer film coating, to aid in vaginal delivery and/or to control the rate of release of the active agents.

The term “vaginal capsule” refers to a solid unit dosage form adapted for insertion into the vagina in which the active agents are encapsulated within a shell. Vaginal capsules include both hard-shelled and soft-shelled capsules, generally known as “hard capsules” and “soft capsules”, respectively. Typically, a hard capsule will contain the active agents and any excipients in the form of a dry powder or granulate. A soft capsule will typically be used for active agents that are dissolved and/or dispersed in an oil.

In embodiments, the composition is provided in the form of a vaginal suppository. In one embodiment, the composition will take the form of a vaginal ovule containing the active agents in a pessary base which includes one or more pharmaceutically acceptable excipients.

In one embodiment, the composition contains only two active agents, i.e., the nitroimidazole and boric acid as herein described. For example, it may include a combination of metronidazole and boric acid, or a combination of tinidazole and boric acid.

In other embodiments, more than two active agents are present. For example, the composition may contain three or more (preferably three) active agents. In order to provide a broader spectrum of activity against vaginal infections, for example, it may be advantageous for the composition to additionally include one or more active agents effective against Candida albicans. This is particularly desirable where metronidazole is present since its administration may result in the proliferation of fungal pathogens. An anti-fungal agent active against Candida albicans may be an imidazole, such as miconazole or tioconazole. Any such agents may be used in the form of their pharmaceutically acceptable salts. Where miconazole is used it may be employed in the form of the free base or as a salt such as the nitrate salt. Typically, it will be employed in the form of miconazole nitrate.

Examples of other active agents that may be present in the compositions disclosed herein include econazole, ketoconazole, itraconazole, posaconazole, fluconazole, voriconazole, clotrimazole, ciclopirox, tolnaftate, terbinafine, sertaconazole, nystatin, tavaborole, efinaconazole. undecylenic acid, and oxiconazole. Derivatives and pharmaceutically acceptable salts of any of these compounds may be employed.

Non-limiting examples of combinations of active agents that may be provided in the compositions for use in the methods disclosed herein include, but are not limited to, metronidazole, miconazole nitrate and boric acid; and tinidazole, tioconazole and boric acid.

It will be understood that the composition containing the active agents will be administered to the subject in a “therapeutically effective amount”, i.e., in an amount that will elicit the biological or medical response of the patient that is being sought by the physician in the treatment of BV as herein described. The “therapeutically effective amount” may depend on factors such as the nature of the particular active agents, the choice of any other actives that may be present, the choice of other non-active components, the severity of the condition, the timing and duration of the treatment, whether the treatment is intended to be therapeutic or prophylactic, etc.

In embodiments, the nitroimidazole and the boric acid are co-administered to the patient per vagina.

The term “co-administration” and “in combination with” are used herein to refer to the delivery of two or more separate chemical entities (e.g., therapeutic agents) in vivo. Co-administration refers to the simultaneous delivery of separate agents; to the simultaneous delivery of a mixture of agents; as well as to the delivery of one agent followed by delivery of a second agent or additional agents. In all cases, agents that are co-administered are intended to work in conjunction with each other, i.e. these will be present together in the vagina to achieve the desired therapeutic and/or prophylactic effect.

In embodiments, the therapeutic agents are in the same composition or unit dosage form. In other embodiments, the therapeutic agents are in separate compositions or unit dosage forms. In certain embodiments, a first agent can be administered prior to (e.g., 1 minute, 2 minutes, 3 minutes, 5 minutes, 7 minutes, 10 minutes, 12 minutes, 15 minutes, or the like before), or concomitantly with the administration of a second therapeutic agent.

“Concomitant administration” of two or more therapeutic agents means administration of the agents at such time that both will have a therapeutic effect. Such concomitant administration may involve concurrent (i.e. at the same time), prior, or subsequent administration of one agent with respect to the administration of a second agent.

In pharmaceutical dosage forms, the compounds may be administered in the form of their pharmaceutically acceptable salts, or they may also be used alone or in appropriate association, as well as in combination, with other pharmaceutically active compounds.

The nitroimidazole active against G. vaginalis may be present in the composition in an amount from 4 to 40 wt. %, preferably from 5 to 35 wt. % (based on the total weight of the composition).

In one embodiment, a high concentration of the nitroimidazole may be employed, for example a concentration that exceeds that conventionally used in the treatment of vaginal infections, such as bacterial vaginosis. Where the nitroimidazole is metronidazole, for example, its concentration may range from 15 to 40 wt. %, preferably from 20 to 35 wt. %, e.g., from 20 to 30 wt. % (based on the total weight of the composition). In one embodiment, the concentration of metronidazole may be 30 wt. % (based on the total weight of the composition). Where the nitroimidazole is tinidazole, its concentration may range from 4 to 20 wt. %, preferably from 5 to 15 wt. %, e.g., from 6 to 12 wt. % (based on the total weight of the composition).

In embodiments, the nitroimidazole is provided in a vaginal suppository and is present in an amount from 100 to 1,000 mg, preferably from 150 to 900 mg (per suppository). In embodiments, the nitroimidazole is metronidazole and is present in an amount from 500 to 1,000 mg, preferably from 550 to 950 mg, more preferably from 600 to 900 mg, yet more preferably from 650 to 850 mg, e.g., from 700 to 800 mg (per suppository). An amount of 750 mg metronidazole per suppository (for example, in an overall suppository weight of 2,500 mg) is particularly preferred. In embodiments, the nitroimidazole is tinidazole and is present in an amount from 100 to 400 mg, preferably from 150 to 300 mg (per suppository).

Though discussed herein in exemplary methods and formulations using a single nitroimidazole compound, mixtures of two or more different nitroimidazole compounds are also contemplated. Any proportional mixtures may be used, for instance where a mixture of two nitroimidazole compounds (e.g., selected from metronidazole, tinidazole, nimorazole, dimetridazole, pretomanid, ornidazole, megazol, azanidazole, secnidazole, and benznidazole) is used, one may be present in 1-99% while the other is present in 99-1% of the total amount of nitroimidazole compound as described herein. Other proportions of two nitroimidazoles used together include 1/99, 3/97, 5/95, 10/90, 15/85, 20/80, 25/75, 30/70, 35/65, 40/60, 45/55, and 50/50. For instance, a dosage amount that contains 100 mg of nitroimidazole compound(s) may contain as little as 1 mg of one type of nitroimidazole and as much as 99 mg of a second type of nitroimidazole—so the total amount of nitroimidazole compounds present in the formulation is 100 mg. However, it is understood (and will be recognized by those of skill in the art) that different nitroimidazoles are useful in different individual dosages—such that NI 1 may be potent and useful at 100 mg (when used alone), while NI 2 may be potent and useful at 200 mg (when used alone; in a 50/50 mixture of NI 1 and NI 2, the relevant potency may be taken into account—such that a “50/50” mix of this exemplar NI 1 and NI 2 would include 50 mg of NI 1 and 100 mg of NI 2 (that being half the effective dosage of each). Similarly, mixtures of three or more nitroimidazole compounds are also contemplated; the proportions can vary in different mixes.

The method of preparation for a mixed-nitroimidazole preparation would be as describe herein (e.g., as per Example 1), where each of the active compounds (Nitroimidazole (NI) 1, NI 2 and Boric Acid) are added sequentially into the melted suppository mass Ovucire® 3460. By way of example, mixtures of Metronidazole and Tinidazole are contemplated, which are formulated to be administrated in the same composition with (or otherwise concurrently with) Boric Acid.

In an embodiment, the amount of boric acid present in the composition will be effective to disrupt, remove or detach at least part of the bacterial biofilm from the vaginal mucosa. In one embodiment, it will be effective to eradicate the biofilm. As used herein, the term “bacterial biofilm” means a community of bacteria which are contained within an extracellular polymeric substance (EPS) matrix produced by the bacteria and attached to a body surface such as the vaginal mucosa. In one embodiment, the boric acid is present in an amount of from 10 to 30 wt. %, preferably from 10 to 25 wt. % (based on the total weight of the composition).

In embodiments, boric acid is provided in a vaginal suppository and is present in an amount from 300 to 900 mg, preferably from 350 to 850 mg, more preferably from 400 to 800 mg, yet more preferably from 450 to 750 mg, e.g., from 500 to 700 mg (per suppository). An amount of 600 mg boric acid per suppository (for example, in an overall suppository weight of 2,500 mg) is particularly preferred.

In embodiments, the imidazole is provided in an amount of from 2 to 10 wt. %, preferably from 4 to 8 wt. % (based on the total weight of the composition).

In embodiments, the imidazole is provided in a vaginal suppository and is present in an amount from 100 to 300 mg, preferably from 150 to 250 mg, e.g., 200 mg (per suppository). In embodiments, the imidazole is miconazole nitrate and the miconazole nitrate is used in an amount from 100 to 300 mg, preferably from 150 to 250 mg, e.g., 200 mg (per suppository). In embodiments, the imidazole is tioconazole and the tioconazole is used in an amount from 100 to 300 mg, preferably 100 or 200 mg per suppository.

The total amount of active agent (i.e., the nitroimidazole, boric acid and, where present, the imidazole) may range from 15 to 50 wt. %, preferably from 20 to 45 wt. %, for example from 25 to 40 wt. % (based on the total weight of the composition). Where the nitroimidazole is metronidazole, the total amount of active agent in the composition will generally be higher than when the nitroimidazole is tinidazole. For example, in the case where the nitroimidazole is metronidazole, the total amount of active agent in the composition may in embodiments be in the range from 25 to 50 wt. %, preferably from 30 to 45 wt. %, for example from 32 to 40 wt. % (based on the total weight of the composition). Where the nitroimidazole is tinidazole, the total amount of active agent in the composition may in embodiments be in the range from 15 to 40 wt. %, preferably from 18 to 36 wt. %, for example from 22 to 32 wt. % (based on the total weight of the composition).

Preferred combinations of active agents for use in the compositions and methods disclosed herein are those which demonstrate enhanced (e.g., synergistic) activity in the treatment of bacterial vaginosis relative to the use of any one of the agents alone, for example which demonstrate enhanced (e.g., synergistic) activity against one or more causative agents of bacterial vaginosis, such as Gardnerella vaginalis, relative to the use of any one of the agents alone. Evidence of synergy may include any one of the following: a faster cure rate, cure time or symptom improvement (i.e., improvement in at least one sign or key symptom of bacterial vaginosis); and a reduction in the relapse rate of bacterial vaginosis (i.e., the rate of reappearance of the infection after cessation of the treatment). In the context of the susceptibility of a microorganism to an antimicrobial agent, synergy may be understood with reference to the definition set out by The European Committee for Antimicrobial Susceptibility Testing (EUCAST) of the European Society of Clinical Microbiology and Infectious Diseases (ESCMID) 2000 (see EUCAST Definitive Document E. Def 1.2: Terminology relating to methods for the determination of susceptibility of bacteria to antimicrobial agents. Clin. Microbiol. Infect., 2000, 6(9): 503-508): FICI≤0.5; synergy.

In embodiments, provided herein are compositions in which the active agents are present in synergistically effective amounts. In this regard, the weight ratio of nitroimidazole compound(s) to boric acid will generally be in the range of from 2:1 to 1:3, preferably from 2:1 to 1:2. Where the nitroimidazole is metronidazole, the weight ratio of metronidazole to boric acid may in embodiments be in the range from 1:0.5 to 1:1.5, preferably 1:0.5 to 1:1.2. Where the nitroimidazole is tinidazole, the weight ratio of tinidazole to boric acid may in embodiments be in the range from 1:1 to 1:3.

In one embodiment, provided herein is a method of treatment of bacterial vaginosis in which the method does not include administering to the patient any therapeutic amount of EDTA. In one embodiment, the composition for use in the method of treatment will be substantially free from EDTA. By “substantially free”, it is intended that the composition will contain less than 1 wt. % EDTA, preferably less than 0.5 wt. % EDTA, e.g., 0 wt. % EDTA.

Typically the compositions for use in the methods disclosed herein will take the form of a vaginal suppository or cream, for instance a vaginal suppository.

Where the formulations are provided in the form of a cream, any conventional cream base may be used, e.g., containing oily or waxy materials such as liquid paraffin, white petroleum or cetyl alcohol, water and one or more surfactants to produce a water-in-oil emulsion. In embodiments, a bactericide such as benzalkonium chloride is present.

In one embodiment, the compositions are provided in the form of a vaginal ovule. When provided in the form of a vaginal ovule, these include a pessary base containing the active agents. Any known pessary base may be used including both water soluble or water-miscible bases and oleaginous (fatty) bases.

Water soluble or water-miscible bases may, for example, contain glycerinated gelatin or polyethylene glycol (PEG) polymers, Glycerinated gelatin pessaries are gelatinous solids that dissolve or disperse slowly in the mucous secretions of the vagina to provide prolonged release, PEG polymers are miscible with mucous secretions in the vagina. Similar to glycerinated gelatin, they do not melt at body temperature but dissolve to provide a prolonged release. Vaginal ovules of different hardness, dissolution time and melting point can be provided using different PEG polymers either singly or, more typically, in combinations of two or more molecular weights in varying proportions. Non-limiting examples of combinations of PEG polymers that may be used in pessary bases include: 30% PEG 1450: 70% PEG 8000; 95% PEG 1000: 5% PEG 3350; 75% PEG 1000: 25% PEG 3350: 60% PEG 300: 40% PEG 8000: 10% PEG 300: 65% PEG 1540: 25% PEG 3350; and 48% PEG 300: 52% PEG 6000.

Oleaginous (fatty) bases include natural, synthetic or semi-synthetic hard fats, and fractionated palm kernel oil. Preferred materials are hard fats which consist mainly of mixtures of the triglyceride esters of the higher saturated fatty acids along with varying proportions of mono- and/or di-glycerides. The choice of different fats and combinations of fats gives rise to a range of melting points and can be selected accordingly. For example, these can be selected to provide a base that melts at body temperature. Special grades may contain additives selected from beeswax, lecithin, polysorbates, fatty acid esters, ethoxylated fatty alcohols and ethoxylated partial fatty glycerides, for example. Natural hard fats that may be used as a pessary base include Theobroma oil or cocoa butter.

The hard fat will typically form the major component of the pessary base. For example, it may be present in an amount of at least 70 wt. % (based on the total weight of the pessary base). Preferably, it will be present in an amount of at least 75 wt. %, more preferably at least 80 wt. %, e.g., at least 85 wt. %.

In one embodiment, the pessary base may additionally include a fatty acid ester derived from a C₈₋₂₂ saturated or unsaturated fatty acid. Examples of saturated fatty acids which may be used to form the fatty acid esters include caproic acid, caprylic acid, capric acid, lauric acid, myristic acid, palmitic acid, stearic acid, arachidic acid and behenic acid. Examples of unsaturated fatty acids which may be used include ricinoleic acid, palmitoleic acid, oleic acid, linoleic acid, linolenic acid and arachidonic acid. In a preferred embodiment, the pessary base includes a fatty acid ester derived from a C₈₋₂₂ unsaturated fatty acid. In preferred embodiments, the unsaturated fatty acid is ricinoleic acid or oleic acid. In preferred embodiments, the fatty acid esters disclosed herein are fatty acid esters of polyhydric alcohols or mixtures thereof. Examples of suitable polyhydric alcohols include glycerol, 1,2-propanediol, and 1,3-propanediol. Fatty acid esters formed from polyhydric alcohols may be mono-, di- or tri-esters. In the case of the di- or tri-esters, the fatty acid components may be the same or different. In a preferred aspect, the polyhydric alcohol is glycerol.

In non-limiting embodiments, the fatty acid esters in which the hydroxy-containing component is a polyhydric alcohol include glyceryl monooleate, glyceryl monolinoleate, glyceryl linolenate, glyceryl monostearate, glyceryl palmitostearate, glyceryl ricinoleate, and mixtures thereof. Glyceryl ricinoleate is particularly preferred.

The fatty acid esters disclosed herein may be commercially available or may be readily synthesized using known esterification methods. Most commercially available fatty acid esters are not 100% pure but will generally contain at least 80%, for example at least 90% by weight of the desired fatty acid ester. Other components which may be present include other fatty acid esters and other fatty acids.

In embodiments, the fatty acid ester or mixture of fatty acid esters is present in an amount of from 3 to 15 wt. %, e.g., from 5 to 10 wt. % (based on the total weight of the pessary base).

In one embodiment, the pessary base may additionally include one or more ethoxylated fatty alcohols. Suitable ethoxylated fatty alcohols include, but are not limited to, Ceteth-20 (polyoxyethylene (20) cetyl ether or polyethylene glycol hexadecyl ether), Steareth-20 (polyethylene glycol octadecyl ether or polyoxyethylene (20) stearyl ether), and mixtures thereof. The amount of ethoxylated fatty alcohol will generally be in the range from 0.5 to 10 wt. % (based on the total weight of the pessary base). Preferably, this component will be present in an amount in the range from 1 to 7 wt. %, for example from 1 to 5 wt. % (based on the weight of the pessary base).

In one embodiment, the pessary base may include the following components: a hard fat formed by reaction of glycerol with C₁₀-C₁₈ saturated fatty acids (i.e., C₁₀-C₁₈ triglycerides); a fatty acid ester formed from glycerol (e.g., glyceryl ricinoleate); and one or more ethoxylated fatty alcohols. For example, the pessary base may include at least 75 wt. % C₁₀-C₁₈ triglycerides; 5 to 10 wt. % glyceryl ricinoleate; and 1 to 5 wt. % ethoxylated fatty alcohols (e.g., Ceteth-20 and/or Steareth-20).

Examples of suitable hard fats include the range of products sold under the trade name Witepsol® (e.g., Witepsol S55, Witepsol W15) by Dynamit Nobel, Slough, England, and those sold by Gattefosse (Westwood, N.J, USA) under the trade names Suppocire® and Ovucire®, Ovucire® WL 3264 consists of a mixture of mono-, di- and tri-glyceride esters of fatty acids (C₁₀ to C₁₈), in which the triester fraction is predominant, and ethoxylated fatty alcohols. Ovucire® 3460 is a hard fat pessary base that consists of a mixture of mono-, di- and triglyceride esters of fatty acids (C₁₀ to C₁₈), the triester fraction being predominant, and esters of ricinoleic (C18:1) acid and ethoxylated fatty alcohols (Ceteth-20/Steareth-20). It has a melting point of from 31.5 to 33.0° C. Ovucire® 3460 is also known as “Hard fat glyceryl ricinoleate Polyoxyl 20 cetostearyl ether” and “Mixture of hard fat, glyceryl ricinoleate and ethoxylated fatty alcohols”. Other known hard fats that may be used include Fattibase® which consists of triglycerides from palm, palm kernel, and coconut oils. Wecobee® is a series of bases. Wecobee® FS, M, R, and S grades are all made from triglycerides of coconut oil but have different melting points. FS has a melting point range of 39.4 to 40.5° C., M has a range of 33.3 to 36.0° C., R has a range of 33.9 to 35.0° C., and S has a range of 38.0 to 40.5° C. Other triglyceride-type bases that may be used include the products sold under the trade names Dehydag®, Hydrokote® and Novata®.

In embodiments, the pessary base contains a surfactant to promote dispersal of the active substances. The surfactant may be a cationic, non-ionic, anionic or amphoteric surfactant although non-ionic surfactants are preferred. Anionic surfactants include salts of long chain alkyl sulphonate esters such as sodium lauryl sulphate, sodium cetostearyl sulphate and sodium tetradecyl sulphate; salts of long chain carboxylic acids such as stearates. Cationic surfactants include quaternary ammonium or pyridinium compounds such as benzalkonium chloride (a mixture of benzyl alkyl dimethyl chlorides, the alkyl chain ranging from C₈ to C₁₈), tetradecyltrimethyl ammonium bromide and cetylpyridinium chloride. Amphoteric surfactants include lauryl 1-carboxy glycine and lecithins such as soya lecithin. Non-ionic surfactants include glycol and glycerol esters such as glyceryl monostearate; macrogol esters and ethers such as cetomacrogol; sorbitan and mannitan esters such as sorbitan tristearate; and polyoxyethylene derivatives of such sorbitan esters, for instance polyoxyethylene (20) sorbitan mono-oleate. The level of surfactant required in the pessary formulation will be readily determined by those skilled in the art and will depend on the specific surfactant and the nature of the pessary base. In embodiments, the surfactant is present in the range from 0.1 to 10 wt. %, preferably 1 to 5 wt. %.

Other components which may be present in the compositions disclosed herein, for example in a cream or vaginal suppository, include local anesthetics, wound healing or skin protectant agents, anti-inflammatory and/or anti-pruritic agents, and bioadhesives,

It may, for example, be advantageous to include one or more local anesthetics in the compositions in order to alleviate the soreness associated with vaginitis. Examples of suitable anesthetics include aptocaine, bupivacaine, butanilicaine, carticaine, cinchocaine, clibucaine, ethyl parapiperidinoacetyl-aminobenzoate, etidocaine, lidocaine (lignocaine), mepivacaine, oxethazaine, prilocaine, pyrrocaine, ropivacaine, tolycaine, vadocaine, benzocaine, pramoxine and mixtures thereof. The anesthetic may also be used in the form of a salt, optionally in combination with the base form whereby to achieve extended release of the anesthetic. The local anesthetic may be used in an amount of 0.1 to 10 wt. %, preferably 1 to 7 wt. %. In one embodiment, the local anesthetic is lidocaine and may be used in the form of its free base (for example in an amount of 1 to 3 wt. %, preferably 1.5 wt. %) or a salt such as its hydrochloride, for example 1.5 to 4 wt. %, preferably 2 wt. %.

One or more wound healing or skin protectant agents may also be present in the compositions. These may be selected from demulcents, absorbents and emollients and include dimethicone (demulcent), allantoin (absorbent), sucralfate and glycerin (absorbent, demulcent and emollient). Examples of other suitable emollients include cocoa butter, white petrolatum and shark liver oil. Dimethicone has been found to be particularly advantageous in facilitating healing of the vaginal mucosa and is therefore particularly preferred for use in the compositions herein described.

In order to counter the inflammation and itching associated with vaginitis, it may be beneficial to include an anti-inflammatory and/or anti-pruritic agent such as hydrocortisone, hydrocortisone acetate, methylprednisolone acepronate, betamethasone valerate, or the like, or a weak topical steroid and/or plant-derived anti-inflammatory bioactive such as bisabolol or chamomile. The compositions may also include chlorophyll as a deodorant.

Natural or synthetic bioadhesive enhancing agents may also be present in the compositions herein described. Examples of such agents include poly(carboxylic acid-containing) based polymers, such as poly(acrylic, maleic, itaconic, citraconic, hydroxyethyl methacrylic, methoxyethyl methacrylic, methoxyethoxyethyl methacrylic or methacrylic) acid which have strong hydrogen-bonding groups, or derivatives thereof such as salts and esters. Appropriate bioadhesives having this form are available commercially (e.g., from Goodrich) as Polycarbophil, e.g., Noveon AA-1, Carbomer (Carbopol), e.g., Carbopol EX165, EX214, 434, 910, 934, 934P, 940, 941, 951, 971, 974P, 980, 981, 1342, and 1382.

Other bioadhesives which may be present include cellulose derivatives such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof, e.g., hydroxypropyl methyl cellulose-E15 (HPMC E-15) or sodium carboxymethyl cellulose-H (Sodium CMC-H). Combinations of two or more cellulose derivatives may also be employed, for example HPMC E-15 and Sodium CMC-H.

Other naturally occurring or synthetic polymers may also be used for their bioadhesive properties, e.g., acacia gums, xanthan gum, guar gum, locust bean gum, tragacanth gums, karaya gum, ghatti gum, cholla gum, psyllium seed gum and gum arabic; clays such as manomorillonite clays, e.g., Veegum, attapulgite clay; polysaccharides such as dextran, pectin, amylopectin, agar, carrageenan, mannan or polygalactonic acid or starches such as hydroxypropyl starch or carboxymethyl starch; lipophilic formulations containing polysaccharides, e.g., Orabase (Bristol Myers Squibb); carbohydrates such as polysubstituted with groups such as sulphate, phosphate, sulphonate or phosphonate, e.g., sucrose octasulphate; polypeptides such as casein, gluten, gelatin, fibrin glue; chitosans which are a natural polycationic copolymer consisting of glycosamine and N-actylglucosamine units (e.g., the chloride salt, lactate or glutamate thereof) or carboxymethyl chitin; glycosaminoglycans such as hyaluronic acid and its derivatives; metals or water soluble salts of alginic acid such as sodium alginate or magnesium alginate; schleroglucan; adhesives containing bismuth oxide or aluminum oxide; atherocollagen; polyvinyl polymers such as polyvinyl alcohols, polyvinylmethyl ethers, polyvinylpyrrolidone, polycarboxylated vinyl polymers (such as polyacrylic acid); polysiloxanes; polyethers; polyalkylene oxides and glycols, e.g., polyethylene oxides and glycols; polyalkoxys and polyacrylamides and derivatives and salts thereof; polyglycolic and polylactic acid homopolymers and copolymers; glycolide and lactide copolymers, e.g., poly-L-(lactide co-glycolide); and polymeric emulsifiers, e.g., Pemulen™ polymeric emulsifiers which are high molecular weight, cross-linked copolymers of acrylic acid and a hydrophobic comonomer.

Where additional bioadhesive components are present, these will preferably be selected from: poly(carboxylic acid-containing) based polymers; tragacanth gums; pectin; carrageenan; chitosan; starches; gelatin; hyaluronic acid and derivatives thereof; cellulose derivatives; polyethylene glycols; and polymeric emulsifiers. Poly(carboxylic acid-containing) based polymers such as polyacrylic acid are especially preferred.

Where any bioadhesive enhancing agents are present, these may be present in the compositions in an amount in the range of from 0.1 to 1 wt. %, preferably from 0.1 to 0.5 wt. %, e.g., 0.1 to 0.3 wt. %.

Other conventional components may also be included in the compositions herein described, for example, preservatives (e.g., propylparaben, methylparaben, phenoxyethanol, etc.), antioxidants (e.g., BHT or BHA), anti-foaming agents (e.g., simethicone emulsion), neutralizing agents, dispersing agents, penetration enhancers, solubilizers, emulsifiers, etc. These components may each be provided in an amount ranging from 0.1 to 15 wt. %, preferably in an amount from 0.1 to 10 wt. %, more preferably from 0.1 to 5 wt. %, e.g., from 0.1 to 1 wt. %.

Anti-foaming agents may be used to suppress foaming during manufacture of the compositions. Particularly suitable for use in this regard are simethicone-containing emulsions, e.g., Simethicone Emulsion, USP which is a non-ionic emulsion containing 30 wt. % simethicone. Solubilizers which may be present include polyvinylpyrrolidones such as plasdone povidone which is a synthetic water-soluble homopolymer of N-vinyl-2-pyrrolidone. Inorganic bases, such as sodium hydroxide, may be present to act as neutralizing agents. Other compounds suitable for this purpose include potassium hydroxide, triethanolaminine, aminomethyl propanol, trisamino- and tetrahydroxypropyl ethylenediamine. Amino acids such as β-alanine and lysine can also be used for neutralization and viscosity modification. Sodium hydroxide may also function as a pH adjuster. Dispersing agents which may be present include propylene glycol. This is also known for its emollient, humectant and viscosity modifier properties. Alternatives to propylene glycol include glycerine, sorbitol, butylene glycol, etc.

The compositions herein described may be manufactured by conventional methods. For example, any creams may be prepared by admixture of the components in an aqueous system including water and a solvent. The solvent should be pharmaceutically acceptable and may be, for example, a C₁₋₆ alcohol, N-methylpyrrolidone, a glycol or a glycol ether (e.g., propylene glycol, 1,3-butylene glycol, dipropylene glycol, diethylene glycol or diethylene glycol monoethyl ether (DGME), an ether (e.g., diethyl ether), or any combination thereof.

The vaginal ovules may be manufactured by conventional methods, for instance by admixture of the active agents in the molten pessary base and pouring the resulting mixture into chilled molds.

The compositions herein described are suitable for use in the treatment of bacterial vaginosis and/or its causative agents. Its causative agents include, but are not limited to, Gardnerella vaginalis and other anaerobic pathogens.

Bacterial vaginosis (BV) is a vaginal infection associated with the presence of Gardnerella vaginalis. Common symptoms include an increased malodorous vaginal discharge that may be white or grey in color, and burning associated with urination. Diagnosis of bacterial vaginosis may be suspected based on symptoms, but many patients with BV are asymptomatic. Patients for treatment in accordance with the methods disclosed herein may be asymptomatic or they may show one or more symptoms of BV. As used herein, the term “bacterial vaginosis” encompasses both symptomatic BV and asymptomatic BV.

Bacterial vaginosis is routinely diagnosed by physical examination and by tests carried out on vaginal secretions. Tests which may be carried out in order to diagnose BV include the following:
Gram stain: a gram stain of vaginal secretions which shows the depletion of lactobacilli and overgrowth of G. vaginalis bacteria usually confirms bacterial vaginosis.
pH test (e.g., using pHydrion® paper): to control bacterial growth the vagina is normally slightly acidic with a pH of 3.8-4.2. A pH greater than 4.5 is indicative of bacterial vaginosis and other inflammatory and infective vaginal processes.
Saline microscopy: some of the vaginal discharge sample is diluted with one or two drops of normal saline and examined under a microscope, first at ×10 magnification, then at ×40. The sample is searched for epithelial cells, blood cells, “clue” cells (i.e., epithelial cells with borders studded or obscured by bacteria), and mobile trichomonads. The presence of “clue” cells is indicative of bacterial vaginosis.
10% KOH whiff test (also known as the “amine test”): a small amount of 10% potassium hydroxide (KOH) is added to some of the vaginal discharge and is sniffed. An amine or fishy odor is considered a positive whiff test and a sign of bacterial vaginosis. The sample is then examined under a microscope for fungal elements.

In clinical practice, bacterial vaginosis may be diagnosed according to the Amsel criteria, which consist of the following:
vaginal pH greater than 4.5.
positive whiff test (amine test).
abnormal vaginal discharge (i.e., a thin, white or yellow, homogenous discharge).
at least 20% “clue” cells on microscopy.
   If three (3) out of the four (4) Amsel criteria are met, bacterial vaginosis is diagnosed.

Alternatively, a diagnosis of bacterial vaginosis may be made based on the observation of certain bacteria from a vaginal swab on Gram strain microscopy. The Gram stain can be scored using the Nugent criteria to give a “Nugent score”. A Nugent score 7 is considered indicative of BV.

In one set of embodiments, the patient for treatment is a female suspected of having bacterial vaginosis. In one embodiment, the patient may be a female exhibiting a malodorous vaginal discharge that is white or grey in color.

In another set of embodiments, the patient for treatment is a female diagnosed as having bacterial vaginosis. In one embodiment, the patient is a female that exhibits 3 out of the 4 Amsel criteria. In another embodiment, the patient is a female that exhibits all 4 Amsel criteria. In another embodiment, the patient is a female that exhibits a Nugent score 7. In another embodiment, the patient is a female that exhibits 3 out of the 4 Amsel criteria and a Nugent score 7. In another embodiment, the patient is a female that exhibits all 4 Amsel criteria and a Nugent score 7.

As used herein, the terms “treatment” or “treating” refer to a reduction in severity of bacterial vaginosis, i.e. regression of the underlying cause of the condition. In some embodiments, treatment includes clinical cure, i.e. elimination of the underlying cause of the condition. Regression of the underlying cause of the condition and clinical cure may be assessed post-treatment using any of the tests described herein in respect of diagnosis. Unless otherwise specified, the terms “treatment” or “treating” as used herein include prophylaxis, i.e., prevention of the occurrence of bacterial vaginosis or prevention of its recurrence.

In some embodiments, the clinical outcome of the treatment will be a female patient that no longer exhibits a malodorous vaginal discharge that is white or grey in color. In another set of embodiments, the clinical outcome of the treatment will be a female patient that exhibits less than 3 out of the 4 Amsel criteria. In another embodiment, the clinical outcome of the treatment will be a female patient that exhibits 2 out of the 4 Amsel criteria. In another embodiment, the clinical outcome of the treatment will be a female patient that exhibits 1 out of the 4 Amsel criteria. In another embodiment, the clinical outcome of the treatment will be a female patient that exhibits 0 out of the 4 Amsel criteria. In another embodiment, the clinical outcome of the treatment will be a female patient that exhibits a Nugent score <7. In another embodiment, the clinical outcome of the treatment will be a female patient that exhibits 0 out of the 4 Amsel criteria and a Nugent score <7.

Advantageously, the treatment method herein described is effective to prevent recurrence of BV post-treatment. In one set of embodiments, the treatment method prevents recurrence of BV for a period of at least 30 days, preferably at least 45 days, more preferably at least 60 days from the start of the treatment.

In one set of embodiments, the patient for treatment is a female having recurrent bacterial vaginosis. As used herein, the term “recurrent bacterial vaginosis” or “RBV” is intended to define bacterial vaginosis in a patient having BV that meets 3 Amsel criteria and that has a history of at least 3 episodes of BV in the previous 12 months. In some embodiments, the patient is a female that is frequently infected with bacterial vaginosis. In one embodiment, a female that is frequently infected with bacterial vaginosis is one who has had at least 4 episodes of BV in the previous 12 months. In one set of embodiments, the patient is a female having recurrent bacterial vaginosis and that has previously undergone treatment with conventional antibacterial therapy. Conventional antibacterial therapy may, for example, include oral or vaginal treatment with clindamycin or metronidazole, or with any other antibiotic.

In some embodiments, the clinical outcome of the treatment will be a female patient that experiences a reduction in further episodes of BV post-treatment. In one embodiment, the outcome of the treatment will be a patient that does not suffer from bacterial vaginosis for a period of up to 30 days after administration of the composition herein described. In other embodiments, the outcome of the treatment will be patient that does not suffer from bacterial vaginosis for a period of up to 45 days, preferably up to 60 days, after administration of the composition.

In one embodiment, the patient for treatment is a female patient having refractory bacterial vaginosis. As used herein, the term “refractory bacterial vaginosis” is intended to define bacterial vaginosis in a patient that does not respond to conventional antibacterial therapy. Conventional antibacterial therapy may include oral or vaginal treatment with clindamycin or metronidazole, or with any other antibiotic. For example, conventional antibacterial therapy may consist of a 5 to 7-day treatment with oral or vaginal metronidazole or clindamycin therapy.

The compositions herein described are also suitable for the treatment of other common causes of vaginitis, including but not limited to, vulvovaginal candidiasis (VVC) and trichomoniasis. In one embodiment, the treatment method is effective in the treatment of BV in a female patient suffering from at least one additional vaginal infection, such as VVC or trichomoniasis. In one embodiment, the treatment is a method for the treatment of mixed vaginitis. As used herein, the term “mixed vaginitis” refers to vaginitis caused by the simultaneous presence of at least two vaginal pathogens that contribute to an abnormal vaginal microbiota.

In one embodiment, the treatment is a method for the prevention of bacterial vaginosis. In this embodiment, the patient may be a female patient that is asymptomatic for bacterial vaginosis and/or that has a negative diagnosis for bacterial vaginosis according to one or more of the clinical criteria set out herein. In one embodiment, the patient is a female that is frequently infected with BV. A female that is frequently infected with bacterial vaginosis may be one who has had at least 4 episodes of BV in the previous 12 months.

In one embodiment, the prophylactic treatment is a maintenance therapy for bacterial vaginosis. As used herein, the term “maintenance therapy” refers to the administration of a therapeutic agent following the main treatment for the disease. In one aspect, the main treatment for the patient is treatment of bacterial vaginosis. The main treatment may be any conventional treatment for BV. In embodiments, the main treatment is a combination treatment as described herein that includes the vaginal administration of a composition including boric acid and a nitroimidazole active against Gardnerella vaginalis, wherein a therapeutically effective amount of the composition is administered.

The precise treatment protocol in accordance with the methods herein described will be dependent on factors such as the severity of the condition, dosage of active agents, whether the treatment is therapeutic or prophylactic, etc.

For therapeutic treatment, the method may include applying the composition to the vagina from once a day to three times a day, preferably once or twice daily. Advantageously, it will be applied once daily. Once daily administration at night (for instance, immediately before bedtime) will generally be preferred in order to maximize contact of the active agents with the pathogens present in the vagina and in order to enhance the efficacy of the treatment. A once daily administration is considered particularly beneficial to aid in patient compliance.

In embodiments, the duration of the therapeutic treatment ranges from 5 to 14 days, for example it may be 5, 6, 7, 8, 9, 10, 11, 12, 13 or 14 days. Advantageously, the treatment period will be short in duration whilst still effective to achieve the desired clinical outcome for the patient and clinician. A treatment period of 5 to 10 days, preferably 6 to 9 days, e.g., 7 days, is considered particularly beneficial.

In one set of embodiments, therapeutic treatment may be carried out once or twice a day for 5 to 14 days, preferably for 5 to 10 days, more preferably 6 to 9 days, e.g., 7 days. In some embodiments, treatment may be carried out once a day for 5 to 14 days, preferably for 5 to 10 days, more preferably 6 to 9 days, e.g., 7 days. A treatment that is carried out once a day for 7 days is particularly preferred.

The compositions disclosed herein may be co-administered with other pharmaceutically active compounds, for example other antibiotics. Advantageously, however, the composition will be applied without other antibiotics (whether simultaneously or sequentially).

Therapeutic treatment in accordance with the methods disclosed herein may be followed by maintenance therapy in order to prevent recurrence of the condition. For example, it may be followed by oral or vaginal metronidazole or clindamycin therapy. In the case where the treatment is followed by maintenance therapy, this may be shorter in duration than conventional maintenance therapy. For example, maintenance therapy may be conducted for a period of 3 months, preferably up to 2 months, e.g., up to 1 month.

As described herein, in one set of embodiments, maintenance therapy may involve administration of the combined boric acid/nitroimidazole composition as herein described. For use in maintenance therapy, any of the dosage forms herein described may be administered to the patient with an appropriate adjustment to the dosage regimen, e.g., the frequency and/or duration of administration. In embodiments, the frequency of administration is reduced in any maintenance therapy, for example it may be reduced to 1 to 3 times a week, e.g., twice weekly. The duration of treatment for the purposes of maintenance therapy may be extended, for example it may be extended up to 1 month, up to 2 months, up to 3 months, up to 4 months, up to 5 months or up to 6 months. In some cases, maintenance therapy may extend for 6-12 months or more.

In a preferred set of embodiments, the therapeutic treatment disclosed herein will not involve any follow-on maintenance therapy.

For prophylactic treatment, the methods disclosed herein may include applying the composition herein described to the vagina from one to three times a week, for example twice a week. Duration of the prophylactic treatment may extend up to several months, for example up to 6 months, up to 12 months, or up to 18 months.

When used to deliver drugs vaginally, the compositions optionally may be administered using a suitable applicator which may be disposable. For example, where the compositions are provided in the form of a liquid or cream, a syringe may be used to deliver this to the desired body cavity (e.g., to the vagina). The syringe may be provided with an appropriate delivery tube or needle.

Provided herein is a device (e.g., a syringe, or a pair of syringes) pre-loaded with at least one dose of a composition (or collectively, a mixture) as herein described. Any such device will generally be adapted to deliver one or more doses of the product in a highly uniform manner. Typically, a single unit dose may include from 1 to 10 grams of the composition, for instance from 2 to 7 grams, e.g., from 3 to 5 grams.

For use in topical application (which includes application directly to the vaginal cavity), the compositions may be packaged in any conventional delivery means such as tubes, containers provided with an actuated plunger, etc.

Further disclosed herein are kits including a composition as herein described and an applicator adapted for delivery of the composition to the vagina.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### EXEMPLARY EMBODIMENTS

1. A method of treating bacterial vaginosis in a female patient in need thereof, including administering vaginally to the patient: 100 to 1,000 mg of nitroimidazole compound(s) active against Gardnerella vaginalis; and 300 to 900 mg boric acid.

2. The method of embodiment 1, wherein the nitroimidazole compound(s) include at least one of: 500 to 1,000 mg metronidazole; or 100 to 400 mg tinidazole.

3. A therapeutic composition, including: 100 to 1,000 mg of nitroimidazole compound(s) active against Gardnerella vaginalis and 300 to 900 mg boric acid, wherein the composition is formulated for vaginal administration.

4. A method of treating bacterial vaginosis in a patient in need thereof, the method including administering vaginally to the patient a composition including: at least one nitroimidazole compound active against Gardnerella vaginalis; and boric acid, wherein a therapeutically effective amount of the composition is administered.

5. The method of embodiment 4, wherein the at least one nitroimidazole includes at least one 5-nitroimidazole or a pharmaceutically acceptable salt thereof.

6. The method of embodiment 4, wherein the at least one nitroimidazole compound includes one or more of metronidazole, tinidazole, nimorazole, dimetridazole, pretomanid, ornidazole, megazol, azanidazole, secnidazole, or benznidazole.

7. The method of embodiment 4, wherein the nitroimidazole compound(s) is present in the composition in an amount from 4 to 40 wt. % based on the total weight of the composition.

8. The method of embodiment 4, wherein the boric acid is present in the composition in an amount of from 10 to 30 wt. % based on the total weight of the composition.

9. The method of embodiment 4, wherein the composition additionally includes an active agent effective against Candida albicans.

10. The method of embodiment 9, wherein the active agent effective against Candida albicans is an imidazole or a pharmaceutically acceptable salt thereof.

11. The method of embodiment 10, wherein the imidazole is selected from miconazole, tioconazole, and pharmaceutically acceptable salts thereof.

12. The method of any one of embodiments 9-11, wherein the active agent effective against Candida albicans is present in the composition in an amount of from 2 to 10 wt. % based on the total weight of the composition.

13. The method of embodiment 4, wherein the composition is substantially free from ethylene diamine tetra acetic acid (EDTA).

14. The method of embodiment 4, wherein the composition is provided in the form of a cream or a vaginal suppository.

15. The method of embodiment 16, wherein the vaginal suppository is a vaginal ovule including a hard fat pessary base.

16. The method of embodiment 14 or embodiment 15, wherein the vaginal suppository contains from 100 to 1,000 mg of the nitroimidazole and from 300 to 900 mg boric acid.

17. The method of embodiment 16, wherein the vaginal suppository contains from 500 to 1,000 mg metronidazole.

18. The method of embodiment 16, wherein the vaginal suppository contains from 100 to 400 mg tinidazole.

19. The method of embodiment 4, wherein the patient is a female having recurrent bacterial vaginosis.

20. The method of embodiment 19, wherein the method is effective to prevent recurrence of bacterial vaginosis in the patient for a period of at least 30 days, preferably at least 45 days, e.g., at least 60 days from the start of the treatment.

21. The method of embodiment 19 or embodiment 20, wherein the treatment is effective to prevent recurrence of bacterial vaginosis in the patient without the need for conventional antibiotic maintenance therapy.

22. The method of embodiment 4, wherein the patient is a female patient having refractory bacterial vaginosis.

23. The method of embodiment 22, wherein the patient is refractory to an FDA-approved treatment for bacterial vaginosis, for example oral or vaginal treatment with clindamycin or metronidazole.

24. The method of embodiment 4, wherein the method includes administration of the composition for a treatment period of: up to 14 days, up to 10 days, or up to 7 days.

25. The method of embodiment 24, wherein the treatment period is 7 days.

26. The method of embodiment 4, wherein the composition is administered once a day.

27. The method of embodiment 4, wherein the composition is provided in the form of a vaginal suppository that contains 750 mg metronidazole and 600 mg boric acid, and wherein the method includes administration of the vaginal suppository once a day for a period of 7 days.

28. A composition including at least one nitroimidazole compound active against G. vaginalis and boric acid for use in a method of treating bacterial vaginosis according to the method of any one of embodiments 1, 2, or 4-27.

29. The composition of embodiment 28, including 100 to 1,000 mg of the nitroimidazole compound and 300 to 900 mg boric acid

30. The composition of embodiment 29, wherein the at least one nitroimidazole compound includes metronidazole

31. The composition of embodiment 30, including 500 to 1,000 mg metronidazole.

32. The composition of embodiment 31, wherein the at least one nitroimidazole compound includes tinidazole.

33. The composition of embodiment 32, including 100 to 400 mg tinidazole.

34. Use of a composition including at least one nitroimidazole compound active against G. vaginalis and boric acid in the manufacture of a medicament for use in a method of treating bacterial vaginosis according to any one of embodiments 1, 2, or 4-27.

35. A kit including: a composition including at least one nitroimidazole compound active against G. vaginalis and boric acid, and an applicator adapted for delivery of the composition to a vaginal cavity of a subject.

36. The kit of embodiment 35, for use in treatment of bacterial vaginosis according to any one of embodiments 1, 2, or 4-27.

37. A pharmaceutical composition formulated for vaginal administration, including: at least one nitroimidazole compound active against Gardnerella vaginalis, boric acid, and at least one pharmaceutically acceptable excipient.

38. The pharmaceutical composition of embodiment 37, wherein the at least one nitroimidazole compound includes a 5-nitroimidazole or a pharmaceutically acceptable salt thereof.

39. The pharmaceutical composition of embodiment 38, wherein the 5-nitroimidazole includes at least one of metronidazole or tinidazole.

40. The pharmaceutical composition of any one of embodiments 37 to 39, wherein one or more of: the at least one nitroimidazole compound is present in the composition in an amount from 4 to 40 wt. % based on the total weight of the composition; the at least one nitroimidazole compound includes one or more of metronidazole, tinidazole, nimorazole, dimetridazole, pretomanid, ornidazole, megazol, azanidazole, secnidazole, or benznidazole; the boric acid is present in the composition in an amount of from 10 to 30 wt. % based on the total weight of the composition; the composition is substantially free from ethylene diamine tetra acetic acid (EDTA); or the composition is provided in the form of a vaginal suppository that contains 750 mg metronidazole and 600 mg boric acid.

41. The pharmaceutical composition of embodiment 40, wherein the composition additionally includes an active agent effective against Candida albicans.

42. The pharmaceutical composition of embodiment 41, wherein the active agent effective against Candida albicans is an imidazole or a pharmaceutically acceptable salt thereof.

43. The pharmaceutical composition of embodiment 42, wherein the imidazole is selected from miconazole, tioconazole, and pharmaceutically acceptable salts thereof.

44. The pharmaceutical composition of 41, wherein the active agent effective against Candida albicans is present in the composition in an amount of from 2 to 10 wt. % based on the total weight of the composition.

45. The pharmaceutical composition of embodiment 40, wherein the composition is provided in the form of a cream or a vaginal suppository.

46. The pharmaceutical composition of embodiment 45, wherein one or more of: the vaginal suppository is a vaginal ovule including a hard fat pessary base; the vaginal suppository contains from 100 to 1,000 mg of the nitroimidazole and from 300 to 900 mg boric acid; the vaginal suppository contains from 500 to 1,000 mg metronidazole; or the vaginal suppository contains from 100 to 400 mg tinidazole.

### Example 1—Ovule Containing Metronidazole and Boric Acid

### Composition of Ovule:

| | | |
|---|---|---|
| Metronidazole | 500 | mg |
| Boric acid | 300 | mg |
| Ovucire ® 3460 | 1,700 | mg |
| Total | 2,500 | mg |

### Method of Preparation:

Ovucire® 3460 was weighed and poured into a beaker. It was placed in a water bath at a temperature of 40-45° C. until all the product had melted. The weighed amount of boric acid was poured into the beaker very slowly and mixed continuously at low speed. When the mixture was homogeneous, the weighed amount of metronidazole was poured into the beaker very slowly. It was mixed continuously at low speed. The temperature of the mixture was then adjusted to 37-38° C. and the mixture poured into ovule molds (casings). The casings were sealed by heat. The final product was cooled to room temperature (25° C.).

### Example 2—Ovule Containing Metronidazole and Boric Acid

### Composition of Ovule:

| | | |
|---|---|---|
| Metronidazole | 500 | mg |
| Boric acid | 600 | mg |
| Ovucire ® 3460 | 1,400 | mg |
| Total | 2,500 | mg |

The ovule was prepared according to the method in Example 1.

### Example 3—Ovule Containing Metronidazole, Miconazole Nitrate and Boric Acid

### Composition of Ovule:

| | | |
|---|---|---|
| Metronidazole | 500 | mg |
| Miconazole nitrate | 200 | mg |
| Boric acid | 300 | mg |
| Ovucire ® 3460 | 1,500 | mg |
| Total | 2,500 | mg |

The ovule was prepared according to the method in Example 1. The weighed amount of micronazole nitrate was added to the mixture together with the metronidazole.

### Example 4—Ovule Containing Tinidazole and Boric Acid

### Composition of Ovule:

| | | |
|---|---|---|
| Tinidazole | 150 | mg |
| Boric Acid | 300 | mg |
| Ovucire ® 3460 | 2,050 | mg |
| Total | 2,500 | mg |

The ovule was prepared according to the method in Example 1. The weighed amount of tinidazole was added to the mixture in place of metronidazole.

### Example 5—Ovule Containing Tinidazole and Boric Acid

### Composition of Ovule:

| | | |
|---|---|---|
| Tinidazole | 200 | mg |
| Boric Acid | 600 | mg |
| Ovucire ® 3460 | 1,700 | mg |
| Total | 2,500 | mg |

The ovule was prepared according to the method in Example 1. The weighed amount of tinidazole was added to the mixture in place of metronidazole.

### Example 6—Ovule Containing Tinidazole and Boric Acid

### Composition of Ovule:

| | | |
|---|---|---|
| Tinidazole | 300 | mg |
| Boric Acid | 600 | mg |
| Ovucire ® 3460 | 1,600 | mg |
| Total | 2,500 | mg |

The ovule was prepared according to the method in Example 1. The weighed amount of tinidazole was added to the mixture in place of metronidazole.

### Example 7—Ovule Containing Tinidazole, Tioconazole and Boric Acid

### Composition of Ovule:

| | | |
|---|---|---|
| Tinidazole | 150 | mg |
| Tioconazole | 100 | mg |
| Boric Acid | 300 | mg |
| Ovucire ® 3460 | 1,950 | mg |
| Total | 2,500 | mg |

The ovule was prepared according to the method in Example 1. The weighed amounts of tinidazole and tioconazole were added to the mixture in place of metronidazole.

### Example 8—Ovule Containing Tinidazole, Tioconazole and Boric Acid

### Composition of Ovule:

| | | |
|---|---|---|
| Tinidazole | 300 | mg |
| Tioconazole | 200 | mg |
| Boric acid | 300 | mg |
| Ovucire 3460 ® | 1,700 | mg |
| Total | 2,500 | mg |

The ovule was prepared according to the method in Example 1. The weighed amounts of tinidazole and tioconazole were added to the mixture in place of metronidazole.

### Example 9—Ovule Containing Metronidazole and Boric Acid

### Composition of Ovule:

| | | |
|---|---|---|
| Metronidazole | 500 | mg |
| Boric acid | 600 | mg |
| Ovucire ® 3460 | 2,700 | mg |
| Total | 3,800 | mg |

The ovule was prepared according to the method in Example 1.

### Example 10—Ovule Containing Metronidazole and Boric Acid

### Composition of Ovule:

| | | |
|---|---|---|
| Metronidazole | 750 | mg |
| Boric acid | 600 | mg |
| Ovucire ® 3460 | 2,450 | mg |
| Total | 3,800 | mg |

The ovule was prepared according to the method in Example 1.

### Example 11—Cream Containing Metronidazole and Boric Acid

### Cream Composition:

| | Weight (g) | Wt. % |
|---|---|---|
| Metronidazole | 0.75 | 15 |
| Boric acid | 0.60 | 12 |
| Polysorbate 60 (Tween 60) | 0.10 | 2 |
| Sorbitan Monostearate (Span 60) | 0.03 | 0.6 |
| Cetyl stearyl alcohol | 0.20 | 4 |
| White petroleum jelly | 0.35 | 7 |
| Liquid petroleum jelly | 0.35 | 7 |
| Water | 2.62 | 52.4 |
| Total | 5 | 100 |

### Method of Preparation:

Oil phase: sorbitan monostearate (Span 60), cetyl stearyl alcohol, white petroleum jelly, and liquid petroleum jelly are added into a beaker and melted while stirring. The mixture is then heated to 70° C.

Aqueous phase: water is heated up to the same temperature as the oil phase (70° C.). Polysorbate 60 (Tween 60) is added into the water and then boric acid is added into the mixture while stirring.

The oil phase is added into the aqueous phase, then it is stirred and homogenized. The mixture is then cooled down to 50° C. Metronidazole is added into the emulsion in 3 equal portions while stirring and homogenized after each addition. The resulting mixture is cooled to room temperature while stirring.

### Example 12—Cream Containing Metronidazole, Miconazole Nitrate and Boric Acid

### Cream Composition:

| | Weight (g) | Wt. % |
|---|---|---|
| Metronidazole | 0.75 | 15 |
| Boric acid | 0.60 | 12 |
| Miconazole Nitrate | 0.20 | 4 |
| Polysorbate 60 (Tween 60) | 0.11 | 2.2 |
| Sorbitan Monostearate (Span 60) | 0.04 | 0.8 |
| Cetyl stearyl alcohol | 0.20 | 4 |
| White petroleum jelly | 0.35 | 7 |
| Liquid petroleum jelly | 0.35 | 7 |
| Water | 2.40 | 48 |
| Total | 5 | 100 |

### Method of Preparation:

Oil phase: sorbitan monostearate (Span 60), cetyl stearyl alcohol, white petroleum jelly, and liquid petroleum jelly are added into a beaker and melted while stirring. The mixture is then heated to 70° C.

Aqueous phase: water is heated up to the same temperature as the oil phase (70° C.). Polysorbate 60 (Tween 60) is added into the water and then boric acid is added into the mixture while stirring.

The oil phase is added into the aqueous phase, then it is stirred and homogenized. The mixture is then cooled down to 50° C. Miconazole nitrate is added in one portion. Then metronidazole is added into the emulsion in three equal portions while stirring and homogenized after each addition. The resulting mixture is cooled to room temperature while stirring.

### Example 13—Stability Studies

Storage stability of the ovules prepared in Examples 1, 5, 6, 9 and 10 was assessed by observation over a 3, 6 or 12-month period. For some of the ovules it was not possible to test beyond 3 or 6 months due to the time available for the testing. Immediately after production (0 months), each ovule was assessed and had the following visual/sensory characteristics: (i) shiny and visually homogenous in composition; (ii) white in color; and (iii) odorless. These characteristics were re-assessed following storage under the following temperature and relative humidity (RH) conditions: (a) 25° C.±2° C./60% RH±5%; and (b) 40° C.±2° C./75% RH±5%.

The results are shown in Tables 1 to 5. In all cases, for the duration of the test period, all ovules retained their original visual/sensory characteristics. Notably, these did not show any sign of discoloration that could indicate a reaction between the boric acid and the imidazole.

TABLE 1
Stability results - Example 1
METRONIDAZOLE OVULE
(500 mg Metronidazole + 300 mg Boric acid)

| MONTH | 25° C. ± 2° C./60% RH ± 5% | 40° C. ± 2° C./75% RH ± 5% |
|---|---|---|
| 3 | Shiny, homogeneous ovule | Shiny, homogeneous ovule |
| | White color | White color |
| | Odorless | Odorless |
| 6 | Shiny, homogeneous ovule | Shiny, homogeneous ovule |
| | White color | White color |
| | Odorless | Odorless |
| 12 | Shiny, homogeneous ovule | Shiny, homogeneous ovule |
| | White color | White color |
| | Odorless | Odorless |

TABLE 2
Stability results - Example 5
TINIDAZOLE OVULE
(200 mg Tinidazole + 600 mg Boric acid)

| MONTH | 25° C. ± 2° C./60% RH ± 5% | 40° C. ± 2° C./75% RH ± 5% |
|---|---|---|
| 3 | Shiny, homogeneous ovule | Shiny, homogeneous ovule |
| | White color | White color |
| | Odorless | Odorless |
| 6 | Shiny, homogeneous ovule | Shiny, homogeneous ovule |
| | White color | White color |
| | Odorless | Odorless |
| 12 | Shiny, homogeneous ovule | Shiny, homogeneous ovule |
| | White color | White color |
| | Odorless | Odorless |

TABLE 3
Stability results - Example 6
TINIDAZOLE OVULE
(300 mg Tinidazole + 600 mg Boric Acid)

| MONTH | 25° C. ± 2° C./60% RH ± 5% | 40° C. ± 2° C./75% RH ± 5% |
|---|---|---|
| 3 | Shiny, homogeneous ovule | Shiny, homogeneous ovule |
| | White color | White color |
| | Odorless | Odorless |
| 6 | Shiny, homogeneous ovule | Shiny, homogeneous ovule |
| | White color | White color |
| | Odorless | Odorless |

TABLE 4
Stability results - Example 9
METRONIDAZOLE OVULE
(500 mg Metronidazole + 600 mg Boric Acid)

| MONTH | 25° C. ± 2° C./60% RH ± 5% | 40° C. ± 2° C./75% RH ± 5% |
|---|---|---|
| 3 | Shiny, homogeneous ovule | Shiny, homogeneous ovule |
| | White color | White color |
| | Odorless | Odorless |

TABLE 5
Stability results - Example 10
METRONIDAZOLE OVULE
(750 mg Metronidazole + 600 mg Boric Acid)

| MONTH | 25° C. ± 2° C./60% RH ± 5% | 40° C. ± 2° C./75% RH ± 5% |
|---|---|---|
| 3 | Shiny, homogeneous ovule | Shiny, homogeneous ovule |
| | White color | White color |
| | Odorless | Odorless |

### Example 14—In Vitro Studies Vs. Planktonic Cells of G. vaginalis

Combinations of boric acid with metronidazole or tinidazole were tested in vitro against planktonic bacteria belonging to the species G. vaginalis (ATCC strain 14018), considered among the likely causal pathogens responsible for bacterial vaginosis. The efficacy of the combination treatment was investigated using a checkerboard assay as described by Algburi et al. (Antimicrob Agents, Ch. 61: e00650-17, 2017).

### Method:

G. vaginalis ATCC 14018 was treated with a combination of boric acid with either metronidazole or tinidazole. A 24 h culture of G. vaginalis was diluted to ˜10⁶ CFU/ml. Each agent was diluted two-fold with sBHI (Brain heart infusion+3% horse serum) broth into two separate 96-well non-tissue culture microplates. From each dilution of boric acid, 50 μl was added horizontally over 50 μl of metronidazole or tinidazole. Then, 100 μl of the bacterial suspension (10⁶ CFU/ml) was separately added to each well. The MIC of each combination was determined after 24 h incubation anaerobically at 37° C. Wells that showed no growth after 24 h incubation were then used in the calculation of FIC values.

The fractional inhibitory concentration (FIC) is defined as the concentration that kills when used in combination with another agent divided by the concentration that has the same effect when used alone (see Hall et al., J Antimicrob Chemoth. 11: 427-433, 1983; and Krogstad et al., Antibiotics in Laboratory Medicine, 1986, 557-578). The FIC index (FICI) for the combination of two agents is the sum of their individual FIC values. By convention, the FIC values of the most effective combination are used in calculating the FICI. The European Committee for Antimicrobial Susceptibility Testing (EUCAST) of the European Society of Clinical Microbiology and Infectious Diseases (ESCMID) 2000 adopts the following definitions to determine the susceptibility of bacteria to an antimicrobial agent (see EUCAST Definitive Document E. Def 1.2: Terminology relating to methods for the determination of susceptibility of bacteria to antimicrobial agents. Clin. Microbiol. Infect., 6(9): 503-508, 2000):
FICI value ≤0.5; synergy
0.5<FICI value ≤1; additive effect
1<FICI value <2; indifferent effect
FICI ≥2; antagonistic effect

FICI is defined herein according to EUCAST (Clin. Microbiol. Infect., 6(9): 503-508, 2000).

### Results:

Boric acid was found to have a minimal inhibitory concentration (MIC) of 1.25 μg/ml against G. vaginalis 14018, while the MICs for metronidazole and tinidazole were found to be 6.25 μg/ml, and 1.5625 μg/ml, respectively.

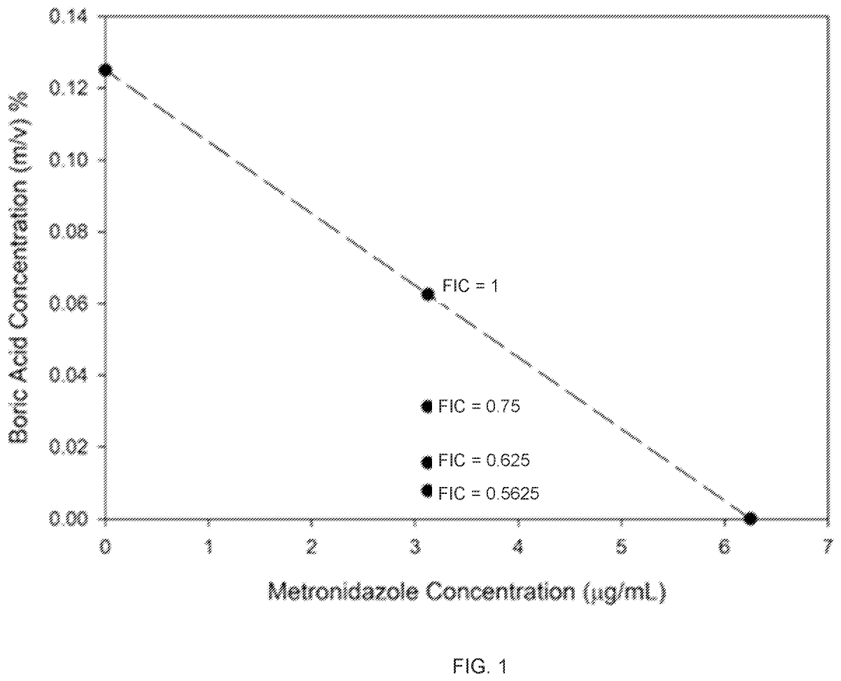

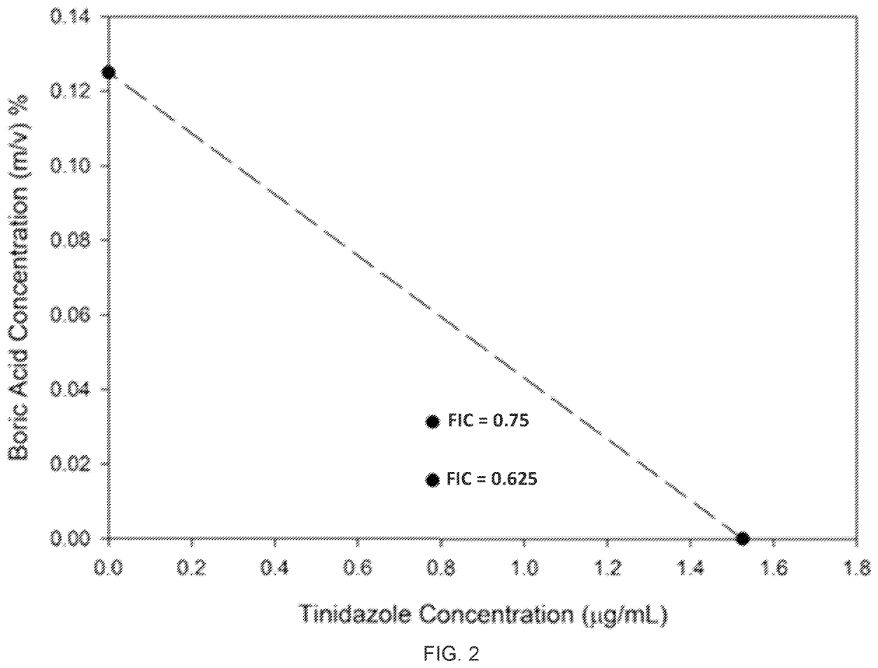

Isobolograms were used to compare the MIC values of each antimicrobial alone to its MIC value in combination with another antimicrobial. For a discussion of using isobolograms in drug combination analysis, see, e.g., Tallarida (J Pharmacol Exp Ther. 319(1):1-7, 2006, doi: 10.1124/jpet.106.104117). FIG. 1 is an isobologram for the combination treatment of boric acid and metronidazole. FIG. 2 is an isobologram for the combination treatment of boric acid and tinidazole. In the Isobolograms, the point on the x axis refers to the MIC value of the first antimicrobial, with the coordinates (0, x), and the point on the y axis represents the MIC value of the second antimicrobial, with the coordinates (y, 0), when the antimicrobials are used alone. The two MIC values are connected by a dashed line (50). The MIC values of each antimicrobial combination are plotted as dots on the graph. Results are expressed according to the locations of these dots relative to the line that connects the MIC values of the first and second antimicrobials. When the MIC values are located under the line, the combination of the two antimicrobials shows synergy, but when these dots of interaction are above the line, the combination of the two antimicrobials shows antagonism against the tested microorganism. An additive effect is observed when these dots are located on the line. The isobolograms confirm that the combinations of boric acid and either metronidazole or tinidazole are synergistic.

The differences between the isobolograms and the FICI values with respect to synergy vs. an additive effect is dependent on the conventions of the two different methods and the designation of an additive effect vs. synergy is arbitrary. In effect, isobolograms are a qualitative means of displaying potential synergy, whereas FICI values provide a means of quantifying synergy, and also introduce a means of measuring either an additive effect or indifference. A combination of the isobolograms overlaid with FICI data represents a means of conveying synergy data in the most comprehensive manner. Whilst the combination of boric acid with metronidazole or tinidazole did not display synergy against planktonic cells of G. vaginalis according to the above FICI definitions, the additive effect that was seen is beneficial. It may, for example, decrease the required concentrations of antimicrobials and make it more difficult for the treated pathogens to develop resistance. This is especially true when the compounds have different modes of action as is the case with boric acid and metronidazole or tinidazole (Rybak et al., Drugs, 52:390-405, 1996). In addition, the use of a non-conventional antimicrobial compound, either alone or in combination with conventional antibiotics, is not associated with the same level of risk seen with the use of conventional antibiotics alone (i.e., tinidazole or metronidazole) (Algburi et al., Pathog Dis, 73: ftv018, 2015).

### Example 15—In Vitro Studies Vs. Biofilm Formation by G. vaginalis

Combinations of boric acid with metronidazole or tinidazole were tested in vitro in a non-planktonic, biofilm model for inhibition of biofilm formation by G. vaginalis (ATCC strain 14018).

### Method:

Bacterial strain, culture media and growth conditions: From the frozen stock (−80° C.), G. vaginalis ATCC 14018 was inoculated in three culture media and propagated at 37° C. for 48 h. Brain-heart infusion (BHI) broth (Difco BD, Franklin, NJ, USA) supplemented with horse serum 3% (JRH Biosciences, KS) was used to maintain microbial growth. Human blood tissue (HBT) agar (Remel, Lenexa, KS, USA) was used to confirm the purity of the frozen stock. The inoculated broth and HBT agar were incubated anaerobically (10% hydrogen, 5% carbon dioxide and 85% nitrogen) using an anaerobic gloves box (Coy Laboratory Products, Inc., Grass Lake, MI, USA). After the incubation period, the bacterial cells were transferred to BHI broth supplemented with 1% glucose (Fisher Scientific, Waltham, MA, USA) (BHIG), every 24 h twice prior to the initiation of an experiment. In order to provide suitable conditions for G. vaginalis anaerobic growth and to avoid oxidative stress, culture media were pre-incubated in the anaerobic chamber at least overnight before bacterial inoculation.

Stock solutions of antibacterial agents: Antimicrobials were evaluated for their activity against biofilm-associated G. vaginalis. Metronidazole was purchased from Alfa Aesar (Ward Hill, Massachusetts, USA). Metronidazole was prepared as a stock solution of 10 mg mL⁻¹ in ddH₂O. Tinidazole was purchased from TCI America (Portland, OR, USA). Tinidazole was prepared as a stock solution of 10 mg/ml in DMSO. Boric acid was purchased from Fisher Chemical (Hampton, NH, USA). A fresh 1 mg mL⁻¹ boric acid solution in BHIG was prepared at the beginning of each experiment within the anaerobic chamber, and mixed with BHIG that had been in the anaerobic chamber overnight (to avoid oxidative stress). The stock solutions of antimicrobials were sterilized using syringe filter 0.45 μm and kept in the refrigerator for a maximum of 3 weeks. On the day of the experiment, the stock solutions were diluted in the anaerobic chamber (to avoid oxidative stress) with pre-incubated BHIG broth to avoid changing the concentrations of nutrients of growth media.

Determination of minimum biofilm inhibitory concentrations (MICs-B): MIC determination was performed according to Sutyak et al. (Antimicrob Agents, Ch 56: 1756-61, 2012) with minor modifications. Each agent was diluted two-fold with BHIG broth into two separate 96-well non-tissue culture microplates. From each dilution of boric acid, 50 μl was added horizontally over 50 μl of metronidazole or tinidazole. The final volume of antimicrobial agents diluted into the broth was 100 μL in each well. The overnight cell culture at 3±2×10⁸ CFU mL⁻¹ was diluted in BHIG to a final concentration of 5×10⁶ CFU mL⁻¹. From the diluted bacterial cells, 100 μL was transferred into the wells containing predetermined concentrations of antimicrobials. Sterile sealing tape (Thermo Fisher Scientific, Rochester, NY) was used to prevent evaporation. The 96-well plate was then incubated anaerobically at 37° C. for 48 h. After incubation, the biofilm was stained using 0.1% crystal violet as described by Algburi et al. (Antimicrob Agents, Ch. 61: e00650-17, 2017); Weeks et al. (Pathog Dis 77(8):ftz059, 2019; doi: 10.1093/femspd/ftz059).

Biofilm staining using CV: Biofilm staining using CV was performed as described by Borucki et al. (Appl Environ Microbiol, 69(12):7336-42, 2003, doi: 10.1128/AEM.69.12.7336-7342.2003) with minor modifications. Briefly, after counting of the non-adherent cells, the biofilm was fixed at 60° C. for 60 minutes in an inverted position in an incubator (New Brunswick Scientific Co., Inc., NJ, USA). To each well, 125 μl of 0.1% CV was added and left at room temperature for 20 minutes. Each well was then rinsed three or four times with 200 μl of ddH₂O and left for 15 minutes to dry at room temperature. To solubilize the CV-stained biofilm, 200 μl of 95% ethanol in water was added, and the microplate was incubated at 4° C. for 30 minutes. One-hundred-microliter samples of solubilized CV were then transferred to a new flat-bottomed 96-well microplate. The absorbance of each sample was measured using a plate reader at 595 nm (model 550; Bio-Rad Laboratories, Hercules, CA, USA). The MIC-B was defined according to Clinical and Laboratory Standards Institute guidance as the lowest concentration of antimicrobial in wells with absorbance A₅₉₅ equal to or less than 20% of the control's mean absorbance (bacterial growth without antimicrobial addition).

Checkerboard assay for antimicrobial combinations against pre-formed biofilms of G. vaginalis ATCC 14018: To evaluate the potential effectiveness of the boric acid in combination with metronidazole/tinidazole against biofilm-associated G. vaginalis ATCC 14018 cells, a checkerboard assay was performed as described by Algburi et al. (Pathog Dis, 73(5):ftv018, 2015, doi: 10.1093/femspd/ftv018) with minor modifications. For biofilm formation, the overnight culture of G. vaginalis ATCC 14018 was diluted to approximately 107 CFU/ml, and 200 μl was transferred to a 96-well tissue culture microplate and incubated anaerobically at 37° C. for 24 to 36 h. Following biofilm formation and removal of non-adherent cells by washing the biofilm twice with BHIG, each antimicrobial was diluted 2-fold separately with BHIG broth in two 96-well (deep well) microplates. From each dilution of antimicrobial B, 125 μl was added horizontally over 125 μl of antimicrobial A (see FIG. 1 of Laverty et al., Int J Mol Sci, 12(10):6566-96, 2011, doi: 10.3390/ijms12106566). From each combination, 200-μl samples were added to the biofilm in sequence, and the microplate was incubated for 24 h at 37° C. under anaerobic conditions. The spot plate method was utilized for counting the number of CFU per milliliter and evaluating the bactericidal activity (MBC-B) of each antimicrobial combination against biofilm-associated G. vaginalis ATCC 14018.

Plate counting method: The number of bacteria that survived was expressed in colony-forming units per milliliter (CFU mL⁻¹) and was enumerated using the drop plate method. The methodology was performed as previously described (Herigstad et al., J Microbiol Meth, 44: 121-9, 2001) with a minor modification. The washed biofilm was disrupted by vigorous pipetting with 200 μL of fresh BHIG broth. Six 10-fold dilutions for each well (from 10¹-10⁶ CFU mL⁻¹) were made using pre-incubated fresh BHIG 1% broth. 20 μL of the cell suspension was then transferred from each dilution and spotted in duplicate on BHI agar plates which were then incubated for 72 h at 37° C. under anaerobic conditions. The number of colonies between 2 and 20 CFU per spot was regarded as a quantifiable number.

Checkerboard assay, data analysis: Isobolograms were used to analyze the interactions of metronidazole or tinidazole with boric acid. This method is based on the comparison of the MBC-B value of each individual antimicrobial with its MBC-B value when used in combination. Axis (x) represents the MBC-B of tinidazole or metronidazole (A) with the coordinates (0, x), and axis (y) represents boric acid (B) with the coordinates (y, 0). The two points (A) and (B) are connected by a line (Turovskiy et al., Probiotics Antimicrob Proteins, 3:144-9, 2011). Each MBCs-B value of two combining antimicrobials is represented as a point on the graph. Results are expressed according to locations of MBCs-B points on the line that connects (A) and (B) as follows: when MBCs-B points are located under or above the line, the two combining antimicrobials are synergized or antagonized respectively, against the biofilm-associated G. vaginalis.

Statistics: Each antimicrobial combination was conducted at least three times in duplicate. The results illustrate the average of three experiments.

### Results:

The minimal inhibitory concentration for biofilm is defined as the concentration needed to inhibit 90% of biofilm compared to a positive control (MIC-B₉₀). The MIC-B₉₀ for boric acid was found to be 0.25% m/v (2.5 μg/ml) against G. vaginalis 14018 while the MIC-B₉₀ for both metronidazole and tinidazole was 25 μg/ml. These concentrations are well above the determined MIC concentrations for each compound against planktonic cells, and so the possibility of quorum quenching as a potential mechanism is unlikely.

Combination treatments for each of metronidazole and tinidazole together with boric acid were effective. Several combinations of boric acid together with the nitroimidazole compounds had fractional inhibitory concentration indices (FICI values) for biofilm inhibition (FICI-B₉₀, using 90% inhibition as the cutoff value) ≤0.5, which is indicative of synergy.

Tables 6 and 7 show the data for the checkboard assay as a whole to show the exact concentrations for the different FICI-B₉₀ values, as well as the percentage of biofilm mass for every combination tested. FIC values were determined from those combinations of antimicrobials that fall below the individual MIC-B₉₀ values (shown underlined in the tables). MIC-B₉₀ and FICI₉₀ values were defined as those wells/combinations where the biofilm mass after treatment was <10% of the positive control (these wells are marked with * in the tables). FICI values were calculated as explained above, and the designation synergy vs. additive effect was determined based on the EUCAST definitions as outlined above (i.e. a synergistic effect (SynE) is observed when FICI value ≤0.5; an additive effect (AddE) when 0.5<FICI value ≤1; an indifferent effect (IndE) when 1<FICI value <2; and an antagonistic effect (AntE) when FICI value ≥2).

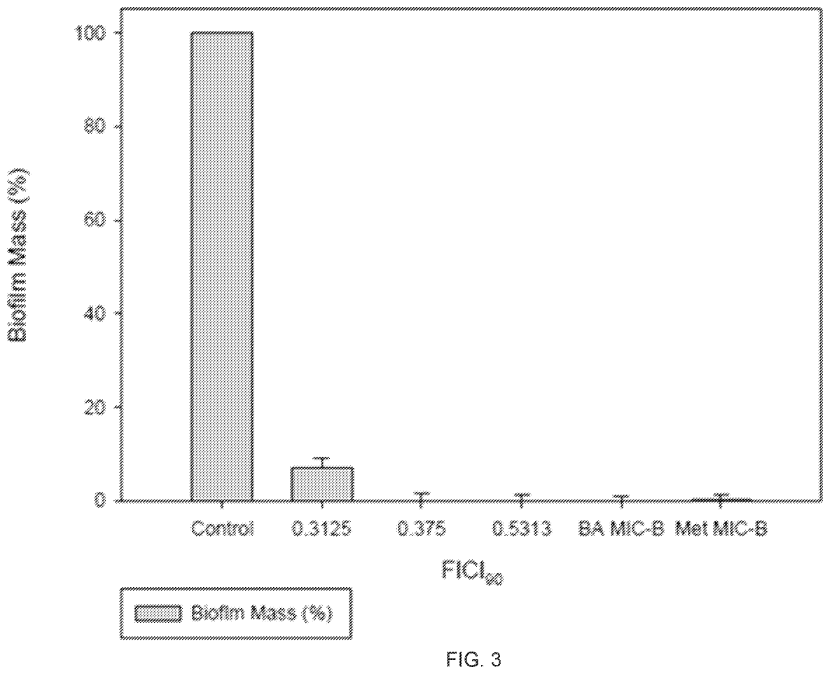

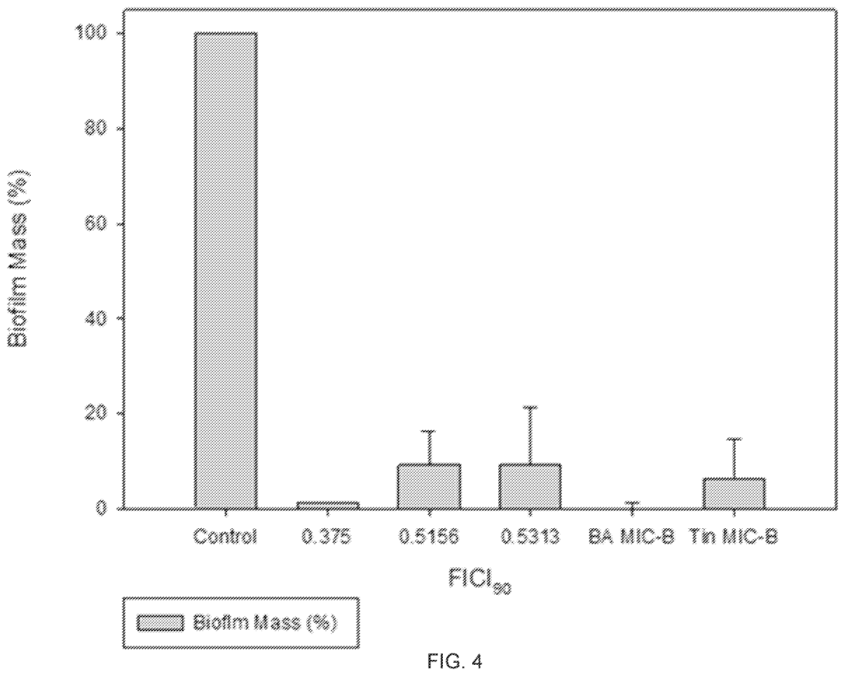

Based on these definitions, there is one combination of metronidazole and boric acid that shows synergy (FICI value ≤0.5) and one combination of tinidazole and boric acid that shows synergy. These are well F3 in Table 6 (FICI₉₀=0.3125) and well D4 in Table 7 (FICI₉₀=0.375), respectively. The other combinations marked with * in Tables 6 and 7 and shown in FIGS. 3 and 4 are indicative of an additive effect (0.5<FICI value <1). FIGS. 3 and 4 are another representation of this data, with each bar representing a combination marked with * from Tables 6 and 7 and showing the relative biofilm mass as a percentage of the control and the FICI value as calculated from the Tables (calculations not shown).

As an example, in FIG. 4, the first bar after the control has a FICI₉₀ of 0.375 and is taken from well D4 in Table 7, which has a relative biofilm mass of 1%. The calculations for the FICI₉₀ are (3.125/25)+(0.0625/0.25). The underlined concentrations are the MIC-B₉₀ values for the two antimicrobials on their own (3.125 for tinidazole and 0.0625 for boric acid), and the concentrations in bold are taken from well D4 in Table 7.

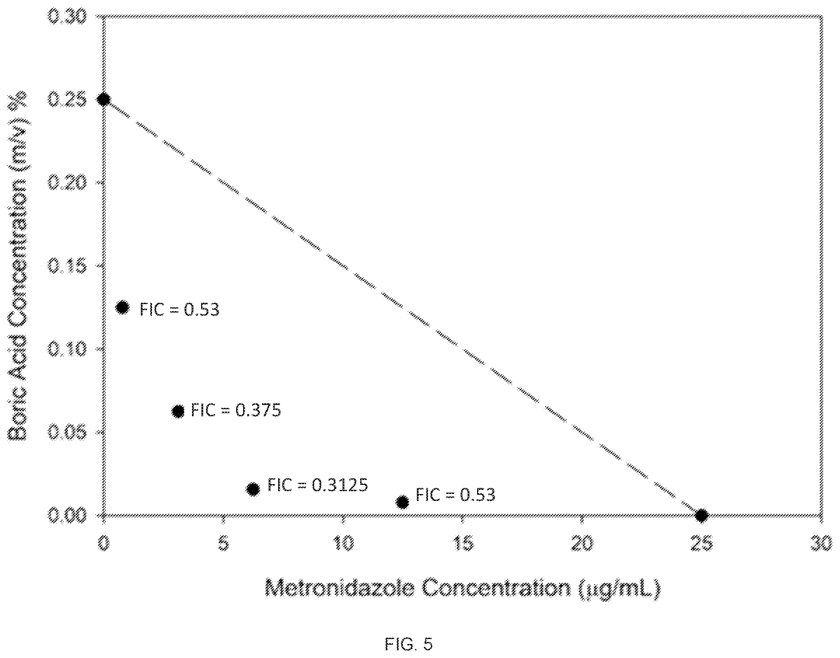

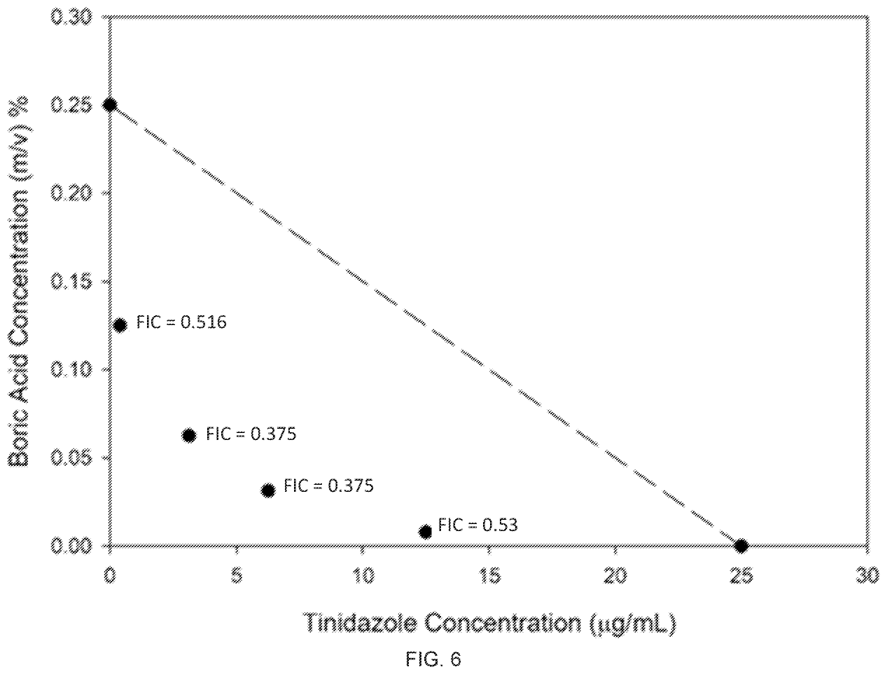

FIGS. 5 and 6 are a further representation of the data. FIG. 5 is an isobologram for the combination treatment of boric acid and metronidazole. FIG. 6 is an isobologram for the combination treatment of boric acid and tinidazole. Calculated FIC values for the combination of boric acid together with metronidazole or tinidazole against biofilm cells of G. vaginalis 14018 are shown in the isobolograms. The isobolograms show that the boric acid and nitroimidazole (metronidazole or tinidazole) combinations were synergistic as all FIC values are located under the dashed line. In respect of both boric acid and nitroimidazole (metronidazole or tinidazole) combinations, two of the FIC values shown in the isobolograms indicated synergy (FICI value ≤0.5) whilst the other two FIC values indicated an additive effect (0.5<FICI value ≤1). As discussed herein, the differences between the isobolograms and the FICI values in respect of synergy vs. additive effect is due to the conventions of the two different methods.

There are observed differences in synergy comparing the planktonic cells, biofilm inhibition, and biofilm killing. This is not unexpected and results from differences in the variables at play for the different experiments. These variables include influence of the biofilm network and the extracellular polymeric substance matrix, differences in the metabolic state of the cells (mostly stationary and much lower activity in a biofilm), the different growth media used, etc. The biofilm model is considered more relevant to the clinical situation in women. Synergy was reflected by a decrease in G. vaginalis induced biofilm mass and a decrease in the number of viable G. vaginalis ATCC 14018 cells within the biofilm, coupled with lower concentrations of metronidazole/tinidazole and boric acid compared to their use alone.

Overall, the in vitro synergy studies provide strong evidence for synergy between metronidazole/tinidazole and boric acid in preventing biofilm formation, which is critical not only in preventing initial colonization by G. vaginalis, but most importantly, may reduce the risk of reinfection and recurrence following treatment. As described herein, recurrence is a significant problem in respect of conventional antibiotic regimens, resulting in increased healthcare costs and the risk of negative health outcomes, such as endometriosis or the increased transmission and acquisition of STIs that may be linked to bacterial vaginosis.

Regarding the efficacy of the combination treatments against preformed biofilms, there is strong evidence for synergy between metronidazole and boric acid, while the tinidazole and boric acid combination has an additive effect. These results show that the tested combinations may be effective in eradicating the biofilms that are a hallmark of bacterial vaginosis.

The results documented herein clearly show that the combination treatment may be used as an effective primary treatment for bacterial vaginosis, and together with the data on biofilm inhibition, may be effective for the treatment and prevention of recurrent bacterial vaginosis, while also greatly reducing the risk for antibiotic resistance development as compared to single drug antibiotic regimens.

TABLE 6
Biofilm mass as a percentage of an untreated control for combination treatments of metronidazole
together with boric acid for the inhibition of biofilm formation by G. vaginalis ATCC 14018.
MIC-B₉₀ concentrations for boric acid and metronidazole alone are underlined. Combinations with
FICI₉₀ values indicative of additive effect or synergy are bold and marked with *.

| | Metronidazole (μg/ml) | | | | | | | | | | |
|---|---|---|---|---|---|---|---|---|---|---|---|
| | 25 | 12.5 | 6.25 | 3.125 | 1.5625 | 0.78125 | 0.390625 | 0.195313 | 0.09765625 | 0 | Boric |
| | 1 | 2 | 3 | 4 | 5 | 6 | 7 | 8 | 9 | 10 | Acid m/v |
| A | 0% | −2% | −2% | −1% | −1% | −2% | −3% | −2% | −3% | −2% | 0.5 |
| B | −1% | −1% | −1% | −2% | −2% | −1% | −3% | −2% | −2% | −1% | 0.25 |
| C | 1% | −1% | −2% | −2% | −2% | 2%* | 22% | 88% | 68% | 85% | 0.125 |
| D | 0% | 1% | −2% | −2%* | 66% | 79% | 79% | 97% | 91% | 95% | 0.0625 |
| E | 0% | 0% | −2% | 14% | 74% | 78% | 81% | 93% | 96% | 92% | 0.03125 |
| F | −2% | 0% | 7%* | 70% | 85% | 89% | 95% | 101% | 100% | 100% | 0.015625 |
| G | 0% | −1%* | 19% | 100% | 90% | 108% | 97% | 106% | 106% | 101% | 0.007813 |
| H | 0% | 11% | 61% | 73% | 88% | 96% | 95% | 123% | 100% | 101% | 0 |

TABLE 7
Biofilm mass as a percentage of an untreated control for combination treatments of tinidazole
together with boric acid for the inhibition of biofilm formation by G. vaginalis ATCC 14018.
MIC-B₉₀ concentrations for boric acid and tinidazole alone are underlined. Combinations with
FICI₉₀ values indicative of additive effect or synergy are bold and marked with *.

| | Tinidazole (μg/ml) | | | | | | | | | | |
|---|---|---|---|---|---|---|---|---|---|---|---|
| | | | | | | | | | | | Boric |
| | 25 | 12.5 | 6.25 | 3.125 | 1.5625 | 0.78125 | 0.390625 | 0.195313 | 0.09765625 | 0 | Acid |
| | 1 | 2 | 3 | 4 | 5 | 6 | 7 | 8 | 9 | 10 | m/v |
| A | 1% | 1% | 1% | 1% | 1% | 1% | −1% | −1% | 0% | −1% | 0.5 |
| B | 1% | 1% | 1% | 1% | 1% | 1% | 0% | −1% | 0% | 0% | 0.25 |
| C | 0% | 1% | 1% | 1% | 2% | 2% | 9%* | 44% | 50% | 63% | 0.125 |
| D | 0% | 1% | 1% | 1%* | 16% | 124% | 81% | 82% | 82% | 88% | 0.0625 |
| E | 1% | 1% | 5%* | 25% | 137% | 122% | 79% | 86% | 88% | 93% | 0.03125 |
| F | 1% | 7% | 28% | 34% | 129% | 127% | 90% | 92% | 94% | 95% | 0.015625 |
| G | 0% | 9%* | 35% | 74% | 119% | 133% | 93% | 97% | 100% | 98% | 0.007813 |
| H | 6% | 15% | 33% | 104% | 133% | 134% | 88% | 92% | 95% | 97% | 0 |

### Example 16—Clinical Studies

Clinical studies are proposed to demonstrate the efficacy and safety of boric acid and metronidazole vaginal ovules in the treatment of recurrent bacterial vaginosis. The drug product will consist of metronidazole (750 mg), boric acid (600 mg) and Ovucire® 3460, a hard fat pessary base delivering enhanced spreadability within the vaginal cavity, designed as a non-irritant with excellent mucosal tolerance. The vaginal ovule will be self-administered via fingertip insertion.

Efficacy will be tested in an initial proof of concept/tolerability study and a Phase 3 study to demonstrate efficacy via statistical significance vs. placebo formulation. The proposed placebo will consist of an identical ovule with boric acid but without the metronidazole component. The intent of the clinical efficacy program is to demonstrate the efficacy of a 7-day treatment with the drug product in both short-term (30 days) and long-term (60 days) test-of-cure endpoints. The design of the studies will be compliant with the August 2019 Guidance Bacterial Vaginosis: Developing Drugs for Treatment. The addition of a long-term primary efficacy endpoint (day 60) following one initial treatment regimen will be unique to the proposed clinical program.

Given that the overall concept is to reduce the occurrence of BV at timepoints beyond day 30, the study populations will be enhanced to enroll only those subjects recently treated for BV with occurrence beyond day 30. The proposed clinical efficacy studies will be as follows:
1. One Phase 2 clinical study of 7 days of treatment (one vaginal ovule per day) vs. placebo. Efficacy will be evaluated at days 30, 60, 90 and 120. Primary endpoints will be at day 30 and day 60; days 90 and 120 will be evaluated as secondary endpoints.
2. One Phase 3 randomized, placebo-controlled study with primary endpoints at day 30 and day 60. The study will be designed and powered based upon the results of the Phase 2 study.

The proposed clinical program is intended to support the approval of a product with efficacy demonstrated out to day 60.

### Example 17—Long-Term Stability Studies

Storage stability of metronidazole in ovules containing metronidazole and boric acid was assessed after storage for approximately 21 months.

After production (essentially according to the method in Example 1, but with 750 mg metronidazole and 600 mg boric acid), the ovules were stored at two different conditions (25° C.±2° C. and 65% RH±5% RH, or at 40° C.±2° C. and 75% RH±5% RH). The stored ovules were analyzed by high-performance liquid chromatography for metronidazole and metronidazole impurity, following published guidelines (Monograph for Metronidazole in the European Pharmacopeia EP10.0, pp3261-3262, Deutscher Apotheker Verlag, 2019).

The results for the two conditions are shown in Tables 8 and 9. These results indicate stability of metronidazole both at 25° C. and 40° C.

TABLE 8
Ovules in storage conditions 25° C. ±
2° C. and 65% RH ± 5% RH

| | Limit | Result |
|---|---|---|
| Metronidazole assay | 100% ± 10% (90%-110%) | 107.2% |
| Metronidazole impurity A | 0.2% | 0.023% |
| Metronidazole total impurity | 0.5% | 0.051% |

TABLE 9
Ovules in storage conditions 40° C. ±
2° C. and 75% RH ± 5% RH

| | Limit | Result |
|---|---|---|
| Metronidazole assay | 100% ± 10% (90%-110%) | 100.1% |
| Metronidazole impurity A | 0.2% | 0.013% |
| Metronidazole total impurity | 0.5% | 0.033% |

### Metronidazole HPLC Assay Method

### HPLC Conditions:

Guard Column: GL Sciences Intertsil ODS-3 4.0×10 mm, 5 μm
Column: Thermo Hypersil BDS C18 4.6×250 mm, 5 μm
Flow rate: 1.0 ml/min
Wavelength: 235 nm
Temperature: 25° C.
Injection Volume: 20 μl
Run time: 7 min
Mobile Phase: Methanol and 20 mM NaH₂PO₄ solution (93:7), (v/v).

Preparation of Standard Solution: Metronidazole working standard (375 mg) was weighed into a 50 ml volumetric flask. Methanol was added, and the metronidazole dissolved in an ultrasonic bath. 1 ml of this solution was transferred by pipette into a 50 ml volumetric flask, where it was diluted to volume with mobile phase and filtered using a 0.45 μm nylon filter.

Preparation of Sample Solution: Pessary was placed into a 100 ml volumetric flask with 50 ml of methanol, then the flask was placed in a water bath (63±2° C.) until the mixture was completely melted. It was then sonicated for 1 minute. The solution was cooled to room temperature in an ice bath, and the mixture diluted to volume with methanol. The mixture was filtered through a blue band filter and the first filtrated 5 ml discharged. 1 ml of the filtrate was transferred by pipette into a 50 ml volumetric flask, then diluted to volume with mobile phase and mixed. This was then filtered through a 0.45 μm nylon filter.

### Calculation:

$$X = \frac{{N}_{met} \times {W}_{met} / 50 \times 1 / 50 \times {W}_{avrg}}{{Std}_{met} \times {W}_{n} / 100 \times 1 / 50 \times 750} \times potence \times 100 = \text{----} Metronidazole$$

Nₘₑₜ: Metronidazole peak area in the Test Solution
Wₘₑₜ: Metronidazole Standard weighed amount (mg)
W_{avrg}: Average of pessary weight (mg)
Wₙ: Weighed sample amount (mg)
Stdₘₑₜ: Metronidazole peak area in the Standard Solution

### Metronidazole Impurity HPLC Assay Method

### HPLC Conditions:

Guard Column: GL Sciences Intertsil ODS-3 4.0×10 mm, 5 μm
Column: Thermo Aquasil C18, 4.6×250 mm, 5 μm
Flow rate: 1.0 ml/min
Wavelength: 315 nm
Temperature: 25° C.
Injection Volume: 10 μl
Run time: 25 min
Buffer solution: Transfer 1.36 g potassium dihydrogen phosphate into a 1000 ml-volumetric flask.
Mobile Phase: Methanol and buffer solution (30:70), (v/v).

Preparation of Sample Solution: 166.7 mg of pessary base was accurately weighed into a 100 ml volumetric flask, and 50 ml of mobile phase added; the flask was then placed in a water bath (63±2° C.) until the mixture was completely melted. The solution was cooled to room temperature in an ice bath, then diluted to volume with mobile phase. The resultant mix was filtered through a blue band filter and the first 5 ml of filtrate discharged, then filtered through a 0.45 μm nylon filter.

Preparation of Standard Solution: 2 ml of this solution was transfer into a 100 ml volumetric flask by pipette, then diluted to volume with mobile phase. 1 ml of this solution was transferred into a 10 ml volumetric flask by pipette, then diluted to volume with mobile phase. The mixture was filtered using a 0.45 μm nylon filter.

Preparation of System Suitability Standard Solution: 5.0 mg of Metronidazole impurity A reference standard was weighed into a 100 ml volumetric flask, and 40 ml of mobile phase was added. The mixture was sonicated, and 10 ml of sample solution added. This was diluted to volume with mobile phase, and 1 ml transferred into a 100 ml volumetric flask by pipette. It was diluted to volume with mobile phase, then filtered using a 0.45 μm nylon filter.

Preparation of Placebo Solution: 116.7 mg of placebo was weighed into a 100 ml volumetric flask, and 50 ml of mobile phase added. The flask was placed in a water bath (63±2° C.) until the mixture was completely melted. The solution was cooled to room temperature in an ice bath, then diluted to volume with mobile phase. The resultant mixture was filtered through blue band filter and the first 5 ml filtrate discharged and filtered through a 0.45 μm nylon filter.

### Calculation:

$$Impurity \% = \frac{Impurity peak area \times {C}_{std}}{Metronidazole Standard Average peak area \times {C}_{sample}} \times 100$$

While the present disclosure describes in detail specific embodiments, it will be understood that various changes and modifications can be made.

### Closing Paragraphs

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the effectiveness of treatment of bacterial vaginosis, for instance.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds, nucleic acid, and amino acids sequences referenced herein that are available in a public database, the information in the database entry is incorporated herein by reference as of the date of an application in the priority chain in which the database identifier for that compound or sequence was first included in the text.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

What is claimed is:

1. A method of treating bacterial vaginosis in a female patient in need thereof, comprising administering vaginally to the patient:
100 to 1,000 mg of nitroimidazole compound(s) active against Gardnerella vaginalis; and
300 to 900 mg boric acid.

2. The method of claim 1, wherein the nitroimidazole compound(s) comprise at least one of:
500 to 1,000 mg metronidazole; or
100 to 400 mg tinidazole.

3. A therapeutic composition, comprising:
100 to 1,000 mg of nitroimidazole compound(s) active against Gardnerella vaginalis and
300 to 900 mg boric acid,
wherein the composition is formulated for vaginal administration.

4. A method of treating bacterial vaginosis in a patient in need thereof, the method comprising administering vaginally to the patient a composition comprising:
at least one nitroimidazole compound active against Gardnerella vaginalis; and
boric acid,
wherein a therapeutically effective amount of the composition is administered.

5. The method of claim 4, wherein the at least one nitroimidazole comprises at least one 5-nitroimidazole or a pharmaceutically acceptable salt thereof.

6. The method of claim 4, wherein the at least one nitroimidazole compound comprises one or more of metronidazole, tinidazole, nimorazole, dimetridazole, pretomanid, ornidazole, megazol, azanidazole, secnidazole, or benznidazole.

7. The method of claim 4, wherein the nitroimidazole compound(s) is present in the composition in an amount from 4 to 40 wt. % based on the total weight of the composition.

8. The method of claim 4, wherein the boric acid is present in the composition in an amount of from 10 to 30 wt. % based on the total weight of the composition.

9. The method of claim 4, wherein the composition additionally comprises an active agent effective against Candida albicans.

10. The method of claim 9, wherein the active agent effective against Candida albicans is an imidazole or a pharmaceutically acceptable salt thereof.

11. The method of claim 10, wherein the imidazole is selected from miconazole, tioconazole, and pharmaceutically acceptable salts thereof.

12. The method of any one of claims 9-11, wherein the active agent effective against Candida albicans is present in the composition in an amount of from 2 to 10 wt. % based on the total weight of the composition.

13. The method of claim 4, wherein the composition is substantially free from ethylene diamine tetra acetic acid (EDTA).

14. The method of claim 4, wherein the composition is provided in the form of a cream or a vaginal suppository.

15. The method of claim 16, wherein the vaginal suppository is a vaginal ovule comprising a hard fat pessary base.

16. The method of claim 14 or claim 15, wherein the vaginal suppository contains from 100 to 1,000 mg of the nitroimidazole and from 300 to 900 mg boric acid.

17. The method of claim 16, wherein the vaginal suppository contains from 500 to 1,000 mg metronidazole.

18. The method of claim 16, wherein the vaginal suppository contains from 100 to 400 mg tinidazole.

19. The method of claim 4, wherein the patient is a female having recurrent bacterial vaginosis.

20. The method of claim 19, wherein the method is effective to prevent recurrence of bacterial vaginosis in the patient for a period from the start of the treatment of:
at least 30 days,
at least 45 days, or
at least 60 days.

21. The method of claim 19, wherein the treatment is effective to prevent recurrence of bacterial vaginosis in the patient without the need for conventional antibiotic maintenance therapy.

22. The method of claim 4, wherein the patient is a female patient having refractory bacterial vaginosis.

23. The method of claim 22, wherein the patient is refractory to an FDA-approved treatment for bacterial vaginosis, for example oral or vaginal treatment with clindamycin or metronidazole.

24. The method of claim 4, wherein the method comprises administration of the composition for a treatment period of:
up to 14 days,
up to 10 days, or
up to 7 days.

25. The method of claim 24, wherein the treatment period is 7 days.

26. The method of claim 4, wherein the composition is administered once a day.

27. The method of claim 4, wherein the composition is provided in the form of a vaginal suppository that contains 750 mg metronidazole and 600 mg boric acid, and wherein the method comprises administration of the vaginal suppository once a day for a period of 7 days.

28. A composition comprising at least one nitroimidazole compound active against G. vaginalis and boric acid for use in a method of treating bacterial vaginosis according to the method of any one of claims 1, 2, or 4-27.

29. The composition of claim 28, comprising 100 to 1,000 mg of the nitroimidazole compound and 300 to 900 mg boric acid

30. The composition of claim 29, wherein the at least one nitroimidazole compound comprises metronidazole

31. The composition of claim 30, comprising 500 to 1,000 mg metronidazole.

32. The composition of claim 31, wherein the at least one nitroimidazole compound comprises tinidazole.

33. The composition of claim 32, comprising 100 to 400 mg tinidazole.

34. Use of a composition comprising at least one nitroimidazole compound active against G. vaginalis and boric acid in the manufacture of a medicament for use in a method of treating bacterial vaginosis according to any one of claims 1, 2, or 4-27.

35. A kit comprising:
a composition comprising at least one nitroimidazole compound active against G. vaginalis and boric acid, and
an applicator adapted for delivery of the composition to a vaginal cavity of a subject.

36. The kit of claim 35, for use in treatment of bacterial vaginosis according to any one of claims 1, 2, or 4-27.

37. A pharmaceutical composition formulated for vaginal administration, comprising:
at least one nitroimidazole compound active against Gardnerella vaginalis, boric acid, and
at least one pharmaceutically acceptable excipient.

38. The pharmaceutical composition of claim 37, wherein the at least one nitroimidazole compound comprises a 5-nitroimidazole or a pharmaceutically acceptable salt thereof.

39. The pharmaceutical composition of claim 38, wherein the 5-nitroimidazole comprises at least one of metronidazole or tinidazole.

40. The pharmaceutical composition of any one of claims 37 to 39, wherein one or more of:
the at least one nitroimidazole compound is present in the composition in an amount from 4 to 40 wt. % based on the total weight of the composition;
the at least one nitroimidazole compound comprises one or more of metronidazole, tinidazole, nimorazole, dimetridazole, pretomanid, ornidazole, megazol, azanidazole, secnidazole, or benznidazole;
the boric acid is present in the composition in an amount of from 10 to 30 wt. % based on the total weight of the composition;
the composition is substantially free from ethylene diamine tetra acetic acid (EDTA); or
the composition is provided in the form of a vaginal suppository that contains 750 mg metronidazole and 600 mg boric acid.

41. The pharmaceutical composition of claim 40, wherein the composition additionally comprises an active agent effective against Candida albicans.

42. The pharmaceutical composition of claim 41, wherein the active agent effective against Candida albicans is an imidazole or a pharmaceutically acceptable salt thereof.

43. The pharmaceutical composition of claim 42, wherein the imidazole is selected from miconazole, tioconazole, and pharmaceutically acceptable salts thereof.

44. The pharmaceutical composition of 41, wherein the active agent effective against Candida albicans is present in the composition in an amount of from 2 to 10 wt. % based on the total weight of the composition.

45. The pharmaceutical composition of claim 40, wherein the composition is provided in the form of a cream or a vaginal suppository.

46. The pharmaceutical composition of claim 45, wherein one or more of:
the vaginal suppository is a vaginal ovule comprising a hard fat pessary base;
the vaginal suppository contains from 100 to 1,000 mg of the nitroimidazole and from 300 to 900 mg boric acid;
the vaginal suppository contains from 500 to 1,000 mg metronidazole; or
the vaginal suppository contains from 100 to 400 mg tinidazole.
